(19) United States
(12) Patent Application Publication
(10) Pub. No.: US 2019/0180856 A1
(43) Pub. Date: Jun. 13, 2019

(54) SYSTEMS AND METHODS FOR OPTIMIZATION OF A BOLUS INSULIN MEDICAMENT DOSAGE FOR A MEAL EVENT

(71) Applicant: Novo Nordisk A/S, Bagsvaerd (DK)

(72) Inventors: Tinna Bjoerk Aradottir, Copenhagen (DK); Henrik Bengtsson, Taastrup (DK); Pete Brockmeier, Copenhagen V (DK); Jonas Kildegaard Pedersen, Vaerloese (DK)

(21) Appl. No.: 16/324,708

(22) PCT Filed: Aug. 14, 2017
(86) PCT No.: PCT/EP2017/070583

§ 371 Date: Feb. 11, 2019

(30) Foreign Application Priority Data
Aug. 18, 2016 (EP) 16184673.8

(51) Int. Cl. G16H 20/17; G16H 50/20

(52) U.S. Cl. CPC G16H 20/17; G16H 50/20

(57) ABSTRACT

Systems and methods for adjusting a short acting dosage for a prospective meal for a subject with a standing regimen are provided. The standing regimen comprises short acting and long acting regimens. Past records are obtained from insulin pens applying the standing regimen. Each record specifies an amount and type of medicament injected, the type being one of short and long acting, and a timestamp. Responsive to the prospective meal at time (t₀), total insulin on board (IOBₜₒₜₐₗ) is calculated as the sum of IOB_{bolus} and IOB_{basal}, with IOB_{bolus} being the total amount of short acting medicament injected, indicated by records having timestamps within a duration of the short acting medicament to t₀, and IOB_{basal} being the total amount of long acting medicament injected, indicated by records having timestamps within the duration of the long acting medicament to t₀. IOBₜₒₜₐₗ serves to calculate the short acting dosage for the meal.

### TECHNICAL FIELD

The present disclosure relates generally to systems and methods for adjusting a short acting insulin medicament dosage for a prospective meal event for a subject with a standing insulin regimen in order to minimize glycaemic risk to the subject.

### BACKGROUND

Type 2 diabetes mellitus is characterized by progressive disruption of normal physiologic insulin secretion. In healthy individuals, basal insulin secretion by pancreatic β cells occurs continuously to maintain steady glucose levels for extended periods between meals. Also in healthy individuals, there is prandial secretion in which insulin is rapidly released in an initial first-phase spike in response to a meal, followed by prolonged insulin secretion that returns to basal levels after 2-3 hours.

Insulin is a hormone that binds to insulin receptors to lower blood glucose by facilitating cellular uptake of glucose, amino acids, and fatty acids into skeletal muscle and fat and by inhibiting the output of glucose from the liver. In normal healthy individuals, physiologic basal and prandial insulin secretions maintain euglycemia, which affects fasting plasma glucose and postprandial plasma glucose concentrations. Basal and prandial insulin secretion is impaired in Type 2 diabetes and early post-meal response is absent. To address these adverse events, subjects with Type 2 diabetes are provided with insulin medicament treatment regimens. Subjects with Type 1 diabetes are also provided with insulin medicament treatment regimens. The goal of these insulin medicament treatment regimens is to maintain a desired fasting blood glucose target level that will minimize estimated risk of hypo- and hyper-glycaemia.

Traditional insulin medicament delivery systems have included the use of pump systems that provide a frequent recurrent dosage of insulin medicament. More recently, additional types of delivery systems have been developed, such as insulin pens, which can be used to self-administer insulin medicament treatment regimens in the form of less frequent insulin medicament injections. A common approach to Type 1 and Type 2 diabetes using such delivery systems is to inject a single short acting insulin medicament (bolus) dosage in a prescribed insulin regimen for the subject in response to or in anticipation of a meal event. In such approaches, the subject injects the short acting insulin medicament dosage shortly before or after one or more meals each day to lower glucose levels resulting from such meals.

However, a problem arises in determining precisely how much short acting insulin medicament should be taken as a single bolus injection for a meal. This problem is subject specific. That is, the optimal amount of the short acting insulin medicament for a particular meal varies from subject to subject, and depends on a number of subject specific factors such as insulin sensitivity, insulin action rate, insulin clearance rate, meal absorption rates, distribution volume, body weight, recent physical exertion of the subject to name a few such factors. As such, failure to inject the correct amount of short acting insulin medicament as a bolus for a meal can result in undesirable changes in glucose levels that may lead to hypo- and/or hyper-glycaemic events.

U.S. Pat. No. 8,140,275 entitled “Calculating Insulin on Board for Extended Bolus Being Delivered by an Insulin Delivery Device,” to Insulet Corporation discloses a system and method used to calculate insulin on board (IOB) for an extended bolus being delivered by an insulin infusion pump. The insulin infusion pump may deliver insulin according to a delivery program that provides different doses of insulin at different times of day, for example, a basal program that provides different basal rates over different time segments. The insulin infusion pump may also deliver bolus doses of insulin, for example, to correct for high blood glucose or in connection with an event, such as a meal, that is likely to affect blood glucose. However, U.S. Pat. No. 8,140,275 does not contemplate the use of insulin pens, rather than insulin pumps. Insulin pens have markedly different characteristics than insulin pumps. For instance, insulin pens can be configured to deliver a long acting basal insulin medicament as one or two injections per day whereas the insulin pump disclosed in U.S. Pat. No. 8,140,275 provides short acting insulin medicament several times day in accordance with a delivery program that specifies a “basal rate.” Moreover, standing insulin regimens based on insulin pen delivery typically make use of a long acting acting insulin medicaments for the basal component of the regimen and a different short acting acting insulin medicament for the bolus component of the regimen. By contrast, the insulin pump disclosed in U.S. Pat. No. 8,140,275 uses the same kind of insulin medicament to account for both basal and bolus component of an insulin medicament regimen. As such, although U.S. Pat. No. 8,140,275 accounts for basal and bolus insulin on board when computing basal rates, the approach taken is not applicable to insulin pen regimens because only a single insulin medicament regimen is contemplated in U.S. Pat. No. 8,140,275 and, moreover, what is sought in U.S. Pat. No. 8,140,275 is an optimal pump rate for delivery of insulin medicament for a meal as opposed to a single bolus amount for the meal.

United States Patent Publication No. 20150306312 entitled “Infusion Devices and Related Methods and Systems for Regulating Insulin on Board” to Metronic Minimed, Inc. similarly discloses systems, devices and methods for delivering a single insulin medicament type to a subject using an insulin pump (also known as a fluid infusion device or infusion pump). As such, United States Patent Publication No. 20150306312 has the same specified above for U.S. Pat. No. 8,140,275.

International Publication WO 15191459 entitled “Insulin Delivery Systems and Methods” to Bigfoot medical, Inc. discloses a system comprising a glucose monitoring device, an insulin pump and a controller configured so that they can communicate with each other using wireless communication channels. The controller is configured to calculate a relative insulin on board value for a specific time by calculating a first value that represents a reference insulin on board value at the specific time, calculating a second value that represents an automated insulin on board value at the specific time, and subtracting one of the first and second values from the other, and wherein the automated insulin on board value represents at least one insulin delivery automatically specified by the computer-based control unit. The publication describes systems and methods relating to pumps and therefore also only one insulin type, namely short-acting insulin.

United States Patent Publication no 20100017141 entitled “Calculating Insulin on Board for Extended Bolus Being Delivered by an Insulin Delivery Device” to Insulet Corporation discloses an insulin on board (IOB) calculation system and method, consistent with embodiments described herein, may be used to calculate insulin on board for an extended bolus being delivered by an insulin infusion pump. In general, the system and method calculates an extended bolus IOB value for the extended bolus, which takes into account the insulin currently on board from the extended bolus and the insulin scheduled to be delivered by the extended bolus over a subsequent time period equivalent to a duration of insulin action. The extended bolus IOB value may be used to calculate a suggested bolus and/or to provide insulin on board information to the user for other purposes. As used herein, “extended bolus” refers to an infusion of a predetermined amount of insulin that includes at least a portion extended over a period of time instead of being delivered immediately. An extended bolus or the extended portion of an extended bolus is generally provided to cover carbohydrate intake (i.e., a meal bolus), although at least a portion of an extended bolus may also be provided to correct a high blood glucose level (i.e., a correction bolus). The duration of time of an extended bolus may vary depending upon various factors such as the nature of the food for which the bolus is being taken (e.g., high fat high protein foods may raise blood sugar for an extended time) and/or the person receiving the insulin (e.g., the ability to digest). The publication describes systems and methods relating to infusion pumps and therefore also only one insulin type, namely short-acting insulin.

International Patent Publication WO 2013/096769 entitled “Systems and Methods for Determining Insulin Therapy for a Patient” to Endotool, LLC may disclose systems and methods for determining subcutaneous insulin therapy for a patient. In one example, information associated with a glucose measurement value of the patient, anticipated nutrition intake for the patient, and a short-acting insulin-on-board for the patient can be received. Based, at least in part, on this information at least one of a short-acting subcutaneous insulin dosage recommendation, a correction subcutaneous insulin dosage recommendation, an intravenous insulin dosage recommendation, a recommended amount of carbohydrates to be administered to the patient, or combinations thereof can be determined. In addition, information indicating a confirmation of a nutrition intake for the patient, and a long-acting insulin-on-board for the patient can be received, and based, at least in part, on this information, a required long-acting subcutaneous or intravenous insulin dosage for the patient can be determined. The short-acting subcutaneous insulin dosage or intravenous insulin dosage recommendation can be adjusted based on a difference between the long-acting insulin-on-board and the required long-acting subcutaneous or intravenous insulin dosage. The medical personnel may administer insulin to the patient subcutaneously using a syringe, or using an infusion pump, for example. The insulin administration may be in accordance with the therapy recommendation provided, for example, by an application server to a workstation and/or input unit. In some examples, the insulin administration may be provided automatically, e.g. by an infusion pump or other device receiving the therapy recommendation provided by the application server and/or workstation. The method may utilize three types of inputs, for example, that can be input per patient by a nurse or other user to a workstation and/or input device as described above. For example, static inputs, glucometer inputs, and/or drug (per oral (PO)) inputs may be used. Drug/PO inputs may include, but are not limited to, any predicted or actual enteral or parenteral carbohydrates taken in by the patient (e.g. including tube feedings), information associated with any dextrose intravenous (IV) drugs administered to the patient, information associated with any steroids administered to the patient, information indicating anticipated or actual activity levels of the patient, information indicating any emesis by the patient, information regarding previous or simultaneous subcutaneous insulin dose(s), and any other subjective information regarding activity of the patient. The workstation may issue orders to administer insulin to the patient depending upon a type of glycemic control methodology ordered by the physician and allowed by the medical administrator of the floor/unit. After administration of subcutaneous insulin to the patient, the nurse may enter a record of administration into the workstation. However, the WO 2013/096769 publication provides no teaching on how to obtain a reliable insulin on board estimate for a subject applying multiple daily injections using manually operated injection devices, e.g., pens or syringes.

Given the above background, what is needed in the art are robust and reliable systems and methods for adjusting a short acting insulin medicament dosage for a prospective meal event for a subject with a standing insulin regimen for treating diabetes, where the standing insulin regimen is administered as both a short acting insulin medicament for meal events and as a long acting insulin medicament for basal treatment. FIG. 7 illustrates this problem. The conventional bolus algorithm applied in FIG. 7 only accounts for a total amount of short acting insulin medicament injected into the subject (IOB_{bolus}) and has no knowledge of a large basal injection, and thus causes hypoglycaemia.

### SUMMARY

The present disclosure addresses the shortcomings identified above. In the present disclosure, systems and methods for providing improved insulin medicament prescription advice are provided. A medical system for estimating a bolus injection is provided that comprises a receiving device adapted to receive (i) data from a first insulin injection device that is related to bolus insulin injection events and corresponding time of bolus injection, and (ii) data from a second insulin injection device related to basal insulin injection events and corresponding time of basal injection. The data from the insulin pens is used by the receiving device to calculate insulin on board based on the bolus and basal data and, from this, calculate a recommended bolus based on the calculated insulin on board. By estimating the total amount of insulin on board, i.e. basal and bolus insulin, bolus calculations are made more accurate and safe. In addition to subtracting a bolus IOB from a calculated meal bolus, basal IOB is also accounted for and subtracted thereby preventing overdosing bolus insulin. FIG. 6 illustrates. The bolus algorithm has knowledge of the total amount of IOB. By taking basal injections into account when determining bolus size, it prevents a hypoglycaemic event by giving a smaller bolus.

WO 2013/096769 publication does at least not disclose a receiving device adapted to receive (i) data from a first insulin injection device that is related to bolus insulin injection events and corresponding time of bolus injection, and (ii) data from a second insulin injection device related to basal insulin injection events and corresponding time of basal injection. Nor does the publication disclose timestamps automatically generated by the respective insulin pen upon occurrence of the respective insulin medicament injection event, or a method of obtaining a first data set from one or more insulin pens used by the subject to apply the standing insulin regimen. The data from the insulin pens is used by the receiving device to calculate insulin on board based on the bolus and basal data and, from this, calculate a recommended bolus based on the calculated insulin on board. Therefore the publication provides no teaching on how to obtain a reliable insulin on board estimate for a subject applying multiple daily injections using manually operated injection devices as pens or syringes.

Accordingly, in one aspect of the present disclosure, a short acting dosage for a prospective meal for a subject with a standing regimen is provided. The standing regimen comprises short acting and long acting regimens. Past records are obtained from insulin pens applying the standing regimen. Each record specifies an amount and type of medicament injected, the type being one of short and long acting, and a timestamp. Responsive to the prospective meal at time t₀, total insulin on board (IOBₜₒₜₐₗ) is calculated as the sum of IOB_{bolus} and IOB_{basal}. Here, IOB_{bolus} is the total amount of short acting medicament injected, indicated by records having timestamps within a duration of action of the short acting medicament to tₒ. IOB_{basal} is the total amount of long acting medicament injected, indicated by records having timestamps within the duration of action of the long acting medicament to t₀. IOBₜₒₜₐₗ is used to calculate the short acting dosage for the meal.

As such, one aspect of the present disclosure provides a device for adjusting a short acting insulin medicament dosage for a prospective meal event for a subject with a standing insulin regimen. The standing insulin regimen comprises a bolus insulin medicament dosage regimen with a short acting insulin medicament and a basal insulin medicament dosage regimen with a long acting insulin medicament. The device comprises one or more processors and a memory. The memory stores a bolus duration of action profile for the short acting insulin medicament that is characterized by a duration of the short acting insulin medicament. The memory also stores a basal duration of action profile for the long acting insulin medicament that is characterized by the duration of the long acting insulin medicament.

The memory further stores instructions that, when executed by the one or more processors, perform a method. In the method, a first data set is obtained from one or more insulin pens used by the subject to apply the standing insulin regimen. The first data set comprises a plurality of insulin medicament records over a time course. Each respective insulin medicament record in the plurality of medicament records comprises (i) a respective insulin medicament injection event including an amount of insulin medicament injected into the subject using a respective insulin pen in the one or more insulin pens, (ii) a respective type of insulin medicament injected into the subject from one of (a) the short acting insulin medicament and (b) the long acting insulin medicament, and (iii) a corresponding electronic timestamp within the time course that is automatically generated by the respective insulin pen upon occurrence of the respective insulin medicament injection event.

In the method, responsive to the prospective meal event associated with the subject at a given time t₀ (e.g., responsive to receiving an indication that the user is going to engage in the prospective meal event), the first data set is used to calculate a total insulin on board IOBₜₒₜₐₗ of the subject using the relation IOBₜₒₜₐₗ=IOB_{bolus}+IOB_{basal}. IOB_{bolus} is calculated from a total amount of short acting insulin medicament injected into the subject indicated by the medicament records in the first data set having timestamps that are within the duration of the short acting insulin medicament to the given time tₒ. IOB_{basal} is calculated from a total amount of long acting insulin medicament injected into the subject indicated by the medicament records in the first data set having timestamps that are within the duration of the long acting insulin medicament to the given time tₒ. IOBₜₒₜₐₗ is then used to calculate the short acting insulin medicament dosage for the prospective meal event for the subject. The short acting insulin medicament dosage for the prospective meal event is then communicated to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event.

In some embodiments, the memory further stores (i) an insulin sensitivity factor for the subject, (ii) a carb to insulin ratio for the subject, and (iii) a target blood glucose level of the subject (BG_{ref}). In such embodiments, the method further comprises obtaining a second data set comprising a plurality of autonomous glucose measurements of the subject and, for each respective autonomous glucose measurement in the plurality of autonomous glucose measurements, a timestamp representing when the respective measurement was made. In such embodiment IOBₜₒₜₐₗ is used to calculate the short acting insulin medicament dosage (Bolus) for the prospective meal event for the subject through the expression:

$$Bolus = \frac{Food ingested in gCHO}{Carb to Insulin ratio} + \frac{BG - {BG}_{ref}}{ISF} - {IOB}_{total}$$

where “Bolus” is the short acting insulin medicament dosage, “Food ingested in gCHO” is estimated based on a type of the prospective meal event, “Carb to Insulin ratio” is the stored carb to insulin ratio of the subject, BG is present blood glucose of the subject obtained from the second data set, BG_{ref} is a target blood glucose of the subject, and ISF is the insulin sensitivity factor of the subject. Non-limiting examples of prospective meal events include “breakfast,” “lunch,” and “dinner.” Further, the memory stores a different “Food ingested in gCHO” value for each type of the prospective meal event. In some embodiments, IOB_{basal} is calculated from a total amount of long acting insulin medicament injected into the subject indicated by the medicament records in the first data set having timestamps that are within the duration of the long acting insulin medicament to the given time tₒ. In such embodiments, each respective amount of long acting insulin medicament injected into the subject indicated by the medicament records in the first data set within the duration of the long acting insulin medicament is discounted by an amount of time between when the respective amount of long acting insulin medicament was injected into the subject and the given time tₒ in accordance with the stored basal duration of action profile for the long acting insulin medicament. In some such embodiments, the first data set indicates that the subject injected the long acting insulin medicament at a single time t₁ within the duration of the long acting insulin medicament to the given time tₒ, and the contribution C_{basal} of the long acting insulin medicament at the time t₁ to IOB_{basal} is calculated as:

$${C}_{basal} = {D}_{basal} * ( \frac{{DIA}_{basal} - {f}_{basal} ( {T}_{basal} )}{{DIA}_{basal}} )$$

where, D_{basal} is a dosage of the long acting medicament injected at time t₁, T_{basal} is the elapsed time between t₁ and t₀, f_{basal}(T_{basal}) is a linear or non-linear function of T_{basal} having a positive value of DIA_{basal}, or less, for any value T_{basal}, and DIA_{basal} is the duration of the long acting insulin medicament obtained from the basal duration of action profile. In some embodiments, f_{basal}(T_{basal}) is T_{basal}, that is f_{basal} is a unity function.

In some embodiments, the first data set indicates that the subject injected the long acting insulin medicament at a time t₁ and a time t₂ within the duration of the long acting insulin medicament to the given time t₀. In such embodiments, the contribution C_{basal1} of the long acting insulin medicament at the time t₁ to IOB_{basal} is calculated as:

$${C}_{basal 1} = {D}_{basal 1} * ( \frac{{DIA}_{basal} - {f}_{basal} ( {T}_{basal 1} )}{{DIA}_{basal}} )$$

where D_{basal1} is a dosage of the long acting medicament injected at time t₁, T_{basal1} is the elapsed time between t₁ and t₀, f_{basal}(T_{basal1}) is a linear or non-linear function of T_{basal1} having a positive value of DIA_{basal}, or less, for any value T_{basal1}, and DIA_{basal} is the duration of the long acting insulin medicament obtained from the basal duration of action profile. In such embodiments, the contribution C_{basal2} of the long acting insulin medicament at the time t₂ to IOB_{basal} is calculated as:

$${C}_{basal 2} = {D}_{basal 2} * ( \frac{{DIA}_{basal} - {f}_{basal} ( {T}_{basal 2} )}{{DIA}_{basal}} )$$

where D_{basal2} is a dosage of the long acting medicament injected at time t₂, T_{basal2} is the elapsed time between t₂ and t₀, and f_{basal}(T_{basal2}) is a linear or non-linear function of T_{basal2} having a positive value of DIA_{basal}, or less, for any value T_{basal2}.

In some embodiments, IOB_{bolus} is calculated from a total amount of short acting insulin medicament injected into the subject indicated by the medicament records in the first data set having timestamps that are within the duration of the short acting insulin medicament to the given time tₒ. Each respective amount of short acting insulin medicament injected into the subject indicated by the medicament records in the first data set within the duration of the short acting insulin medicament is discounted by an amount of time between when the respective amount of short acting insulin medicament was injected into the subject and the given time tₒ in accordance with the stored bolus duration of action profile for the short acting insulin medicament. For instance, in some such embodiments, the first data set indicates that the subject injected the short acting insulin medicament at a single time t₃ within the duration of the short acting insulin medicament to the given time tₒ, and the contribution C_{bolus} of the short acting insulin medicament at the time t₃ to IOB_{bolus} is calculated as:

$${C}_{bolus} = {D}_{bolus} * ( \frac{{DIA}_{bolus} - {f}_{bolus} ( {T}_{bolus} )}{{DIA}_{bolus}} )$$

where D_{bolus} is a dosage of the short acting medicament injected at time t₃, T_{bolus} is the elapsed time between t₃ and t₀, f_{bolus}(T_{bolus}) is a linear or non-linear function of T_{bolus} having a positive value of DIA_{bolus}, or less, for any value T_{bolus}, and DIA_{bolus} is the duration of the short acting insulin medicament obtained from the bolus duration of action profile. In some such embodiments, f_{bolus}(T_{bolus}) is T_{bolus}.

In some embodiments, the first data set indicates that the subject injected the short acting insulin medicament at a first time t₃ and a second time t₄ within the duration of the short acting insulin medicament to the given time tₒ, and the contribution C_{bolus1} of the short acting insulin medicament at the time t₃ to IOB_{bolus} is calculated as:

$${C}_{bolus 1} = {D}_{bolus 1} * ( \frac{{DIA}_{bolus} - {f}_{bolus} ( {T}_{bolus 1} )}{{DIA}_{bolus}} )$$

where D_{bolus1} is a dosage of the short acting medicament injected at time t₃, T_{bolus} is the elapsed time between t₃ and t₀, f_{bolus}(T_{bolus1}) is a linear or non-linear function of T_{bolus1} having a positive value of DIA_{bolus}, or less, for any value T_{bolus1}, DIA_{bolus} is the duration of the short acting insulin medicament obtained from the bolus duration of action profile, and the contribution C_{bolus2} of the short acting insulin medicament at the time t₄ to IOB_{bolus} is calculated as:

$${C}_{bolus 2} = {D}_{bolus 2} * ( \frac{{DIA}_{bolus} - {f}_{bolus} ( {T}_{bolus 2} )}{{DIA}_{bolus}} )$$

where, D_{bolus2} is a dosage of the short acting medicament injected at time t₄, T_{bolus2} is the elapsed time between t₄ and t₀, and f_{bolus}(T_{bolus2}) is a linear or non-linear function of T_{bolus2} having a positive value of DIA_{bolus}, or less, for any value T_{bolus2}.

In some embodiments, successive measurements in the plurality of glucose measurements in the second data set are autonomously taken from the subject at an interval rate of 5 minutes or less, 3 minutes or less, or 1 minute or less. In some embodiments, the device further comprises a wireless receiver, and the first data set is obtained wirelessly from a glucose sensor affixed to the subject.

In some embodiments, the short acting insulin medicament consists of a single insulin medicament having a duration of action that is between three and eight hours or a mixture of insulin medicaments that collectively have a duration of action that is between three and eight hours, and the long acting insulin medicament consists of a single insulin medicament having a duration of action that is between 12 and 24 hours or a mixture of insulin medicaments that collectively have a duration of action that is between 12 and 24 hours.

Another aspect of the present disclosure provides a method for adjusting a short acting insulin medicament dosage for a prospective meal event for a subject with a standing insulin regimen. The memory comprises, at a computer comprising one or more processors and a memory, the memory storing: a standing insulin regimen, where the standing insulin regimen comprises a bolus insulin medicament dosage regimen with a short acting insulin medicament and a basal insulin medicament dosage regimen with a long acting insulin medicament, a bolus duration of action profile for the short acting insulin medicament that is characterized by a duration of the short acting insulin medicament, and a basal duration of action profile for the long acting insulin medicament that is characterized by a duration of the long acting insulin medicament. The memory further stores instructions that, when executed by the one or more processors, perform a method of obtaining a first data set from one or more insulin pens used by the subject to apply the standing insulin regimen.

The first data set comprises a plurality of insulin medicament records over a time course.

Each respective insulin medicament record in the plurality of medicament records comprises (i) a respective insulin medicament injection event including an amount of insulin medicament injected into the subject using a respective insulin pen in the one or more insulin pens, (ii) a respective type of insulin medicament injected into the subject from one of (a) the short acting insulin medicament and (b) the long acting insulin medicament, and (iii) a corresponding electronic timestamp within the time course that is automatically generated by the respective insulin pen upon occurrence of the respective insulin medicament injection event. Responsive to the prospective meal event associated with the subject at a given time tₒ, the first data set is used to calculate a total insulin on board IOBₜₒₜₐₗ of the subject, where the IOBₜₒₜₐₗ is calculated as OBₜₒₜₐₗ=IOB_{bolus}+IOB_{basal}, and where IOB_{bolus} is calculated from a total amount of short acting insulin medicament injected into the subject indicated by the medicament records in the plurality of medicament records having timestamps that are within the duration of the short acting insulin medicament to the given time tₒ, and IOB_{basal} is calculated from a total amount of long acting insulin medicament injected into the subject indicated by the medicament records in the first data set having timestamps that are within the duration of the long acting insulin medicament to the given time tₒ. In such embodiments, the IOBₜₒₜₐₗ is used to calculate the short acting insulin medicament dosage for the prospective meal event for the subject and the short acting insulin medicament dosage for the prospective meal event is communicated to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event.

In a further aspect is provided, a computer program comprising instructions that, when executed by a computer having one or more processors and a memory, perform the method of adjusting a short acting insulin medicament dosage for a prospective meal event for a subject with a standing insulin regimen, the memory comprising:
at a computer comprising one or more processors and a memory:
the memory storing:
   a standing insulin regimen, wherein the standing insulin regimen comprises a bolus insulin medicament dosage regimen with a short acting insulin medicament and a basal insulin medicament dosage regimen with a long acting insulin medicament,
   a bolus duration of action profile for the short acting insulin medicament that is characterized by a duration of the short acting insulin medicament, and
   a basal duration of action profile for the long acting insulin medicament that is characterized by a duration of the long acting insulin medicament,
the memory further storing instructions that, when executed by the one or more processors, perform a method of:
obtaining a first data set from one or more insulin pens used by the subject to apply the standing insulin regimen, the first data set comprising a plurality of insulin medicament records over a time course, each respective insulin medicament record in the plurality of medicament records comprising:
   (i) a respective insulin medicament injection event including an amount of insulin medicament injected into the subject using a respective insulin pen in the one or more insulin pens,
   (ii) a respective type of insulin medicament injected into the subject from one of (a) the short acting insulin medicament and (b) the long acting insulin medicament, and
   (iii) a corresponding electronic injection event timestamp within the time course that is automatically generated by the respective insulin pen upon occurrence of the respective insulin medicament injection event; and
responsive to receiving an indication of the prospective meal event associated with the subject at a given time tₒ:
   using the first data set to calculate a total insulin on board IOBₜₒₜₐₗ of the subject, wherein the IOBₜₒₜₐₗ is calculated using the relation:

IOBₜₒₜₐₗ=IOB_{bolus}+IOB_{basal}
wherein,
   IOB_{bolus} is calculated from a total amount of short acting insulin medicament injected into the subject indicated by the medicament records in the plurality of medicament records having injection event timestamps that are within the duration of the short acting insulin medicament to the given time tₒ, and
   IOB_{basal} is calculated from a total amount of long acting insulin medicament injected into the subject indicated by the medicament records in the first data set having injection event timestamps that are within the duration of the long acting insulin medicament to the given time tₒ;
using the IOBₜₒₜₐₗ to calculate the short acting insulin medicament dosage for the prospective meal event for the subject; and
communicating the short acting insulin medicament dosage for the prospective meal event to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event.

In a further aspect is provided, a computer-readable data carrier having stored thereon the computer program as described above.

### BRIEF DESCRIPTION OF THE DRAWINGS

FIG. 1 illustrates an exemplary system topology that includes a regimen dosage device for adjusting a short acting insulin medicament dosage for a prospective meal event for a subject with a standing insulin regimen, a data collection device for collecting patient data, one or more glucose sensors that measure glucose data from the subject, and one or more insulin pens that are used by the subject to inject insulin medicaments in accordance with the standing insulin regimen, where the above-identified components are interconnected, optionally through a communications network, in accordance with an embodiment of the present disclosure.

FIG. 2 illustrates a device for adjusting a short acting insulin medicament dosage for a prospective meal event for a subject with a standing insulin regimen in accordance with an embodiment of the present disclosure.

FIG. 3 illustrates a device for adjusting a short acting insulin medicament dosage for a prospective meal event for a subject with a standing insulin regimen in accordance with another embodiment of the present disclosure.

FIGS. 4A, 4B, 4C, 4D, and 4E collectively provide a flow chart of processes and features of a device for adjusting a short acting insulin medicament dosage for a prospective meal event for a subject with a standing insulin regimen, where optional elements of the flow chart are indicated by dashed boxes, in accordance with various embodiments of the present disclosure

FIG. 5 illustrates an example integrated system of connected insulin pen(s), continuous glucose monitor(s), memory and a processor for adjusting a short acting insulin medicament dosage for a prospective meal event for a subject with a standing insulin regimen in accordance with an embodiment of the present disclosure.

FIG. 6, top panel (A), illustrates glucose concentration over a 30 hour period during which three meals are ingested with corresponding short acting insulin medicament injections (boluses) and one long acting insulin medicament (basal) injection, whereas FIG. 6, bottom panel (B), shows an estimation of active insulin units at every point in time, for short acting insulin medicament insulin, long acting insulin medicament insulin, and their total, in accordance with an embodiment of the present disclosure.

FIG. 7 illustrates a bolus algorithm that only accounts for a total amount of short acting insulin medicament injected into the subject (IOB_{bolus}) and has no knowledge of a large basal injection, and thus causes hypoglycaemia, in accordance with the prior art.

FIG. 8 illustrates a bolus algorithm that has knowledge of the total amount of insulin on board IOBₜₒₜₐₗ, in which a total amount of short acting insulin medicament injected and a total amount of a total amount of long acting insulin medicament injected is taken into account when adjusting a short acting insulin medicament dosage (bolus size), thereby preventing a hypoglycaemic event by giving a smaller short acting insulin medicament dosage (bolus dosage) in accordance with an embodiment of the present disclosure.

Like reference numerals refer to corresponding parts throughout the several views of the drawings.

### DETAILED DESCRIPTION

The present disclosure relies upon the acquisition of a data set comprising a plurality of insulin medicament records taken over a time course. Each respective insulin medicament record in the plurality of insulin medicament records comprises (i) a respective insulin medicament injection event including an amount of insulin medicament injected into a subject using a respective insulin pen in a set of one or more insulin pens, (ii) a respective type of insulin medicament injected into the subject from one of (a) a short acting insulin medicament and (b) a long acting insulin medicament, and (iii) a corresponding electronic injection event timestamp within the time course that is automatically generated by the respective insulin pen upon occurrence of the respective insulin medicament injection event.

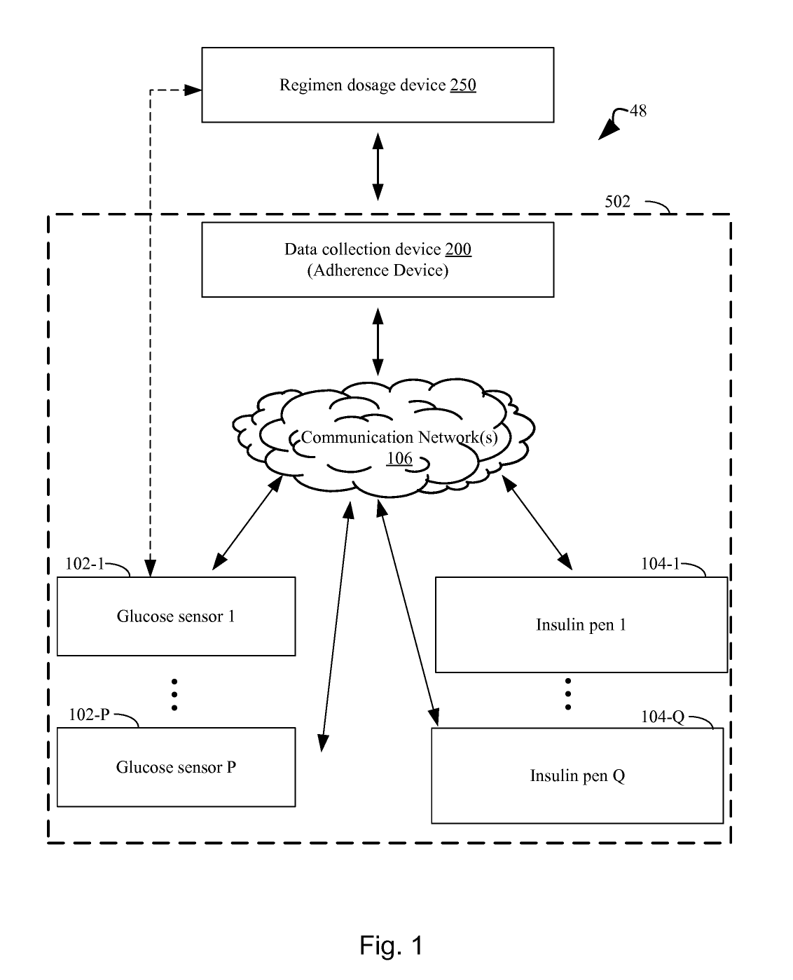

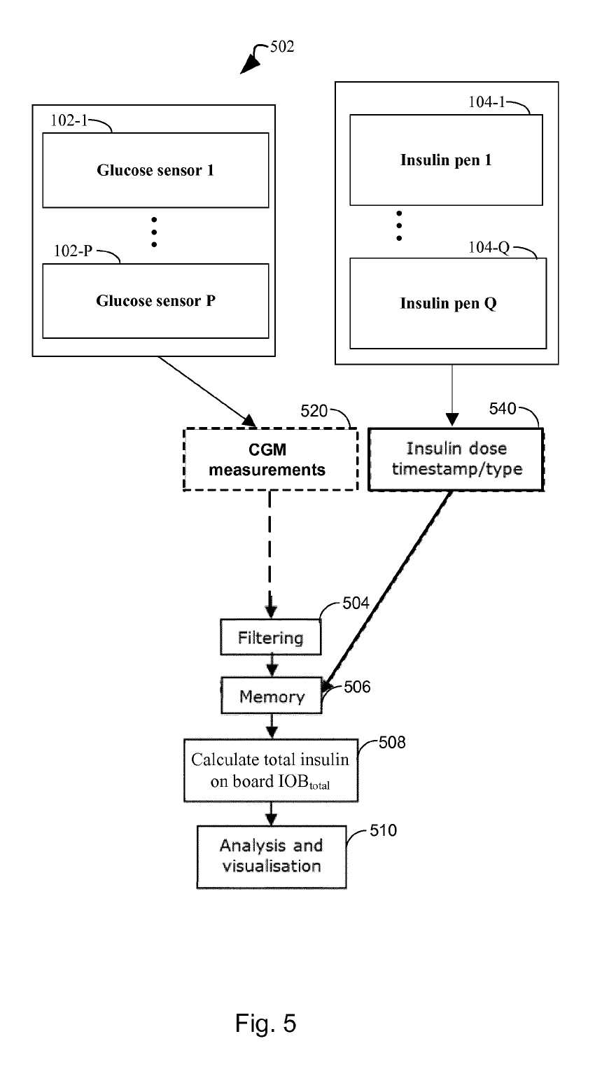

FIG. 1 illustrates an example of an integrated system 502 for the acquisition of such data, and FIG. 5 provides more details of such a system 502. The integrated system 502 includes one or more connected insulin pens 104, one or more glucose monitors 102, memory 506, and a processor (not shown) for optimizing a timing of a short acting insulin medicament dosage in a prescribed insulin regimen for a subject. In some embodiments, a glucose monitor 102 is a continuous glucose monitor.

With the integrated system 502, data from the one or more insulin pens 104, used to apply a standing insulin regimen to the subject, is obtained 540 as a plurality of insulin medicament records. Each insulin medicament record comprises a timestamped event specifying an amount of injected insulin medicament that the subject received as part of the standing insulin medicament dosage regimen. Also, in some embodiments, autonomous timestamped glucose measurements of the subject are obtained 520. In such embodiments, the autonomous glucose measurements are filtered 504 and stored in non-transitory memory 506. The plurality of insulin medicament records of the subject taken over a time course are used to calculate a total insulin on board IOBₜₒₜₐₗ of the subject. In this way, the insulin medicament records are analyzed and visualized (e.g., to adjust a short acting insulin medicament dosage for a prospective meal event for a subject) in accordance with the methods of the present disclosure 510.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein. By the term insulin pen is meant an injection device suitable for applying discrete doses of insulin, where the injection device is adapted for logging and communicating dose related data.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

A detailed description of a system 48 for adjusting a short acting insulin medicament dosage for a prospective meal event for a subject in accordance with the present disclosure is described in conjunction with FIGS. 1 through 3. As such, FIGS. 1 through 3 collectively illustrate the topology of the system in accordance with the present disclosure. In the topology, there is a regimen dosage device adjusting a short acting insulin medicament dosage (210) for a prospective meal event for a subject (“regimen dosage device 250”) (FIGS. 1, 2, and 3), a device for data collection (“data collection device 200”), one or more insulin pens 104 for injecting insulin medicaments into the subject (FIGS. 1 and 5), and optionally one or more glucose sensors 102 associated with the subject (FIGS. 1 and 5). Throughout the present disclosure, the data collection device 200 and the regimen dosage device 250 will be referenced as separate devices solely for purposes of clarity. That is, the disclosed functionality of the data collection device 200 and the disclosed functionality of the regimen dosage device 250 are contained in separate devices as illustrated in FIG. 1. However, it will be appreciated that, in fact, in some embodiments, the disclosed functionality of the data collection device 200 and the disclosed functionality of the regimen dosage device 250 are contained in a single device. In some embodiments, the disclosed functionality of the data collection device 200 and/or the disclosed functionality of the regimen dosage device 250 are contained in a single device and this single device is an insulin pen 104.

Referring to FIG. 1, the regimen dosage device 250 adjusts a short acting insulin medicament dosage for a prospective meal event for a subject. To do this, the data collection device 200, which is in electrical communication with the regimen dosage device 250, receives a plurality of insulin medicament records over a time course, each record comprising (i) an insulin medicament injection event including an amount of insulin medicament injected into the subject using a respective insulin pen 104 in the one or more insulin pens, (ii) a respective type of insulin medicament injected into the subject from one of short and long acting insulin medicament, and (iii) a corresponding electronic injection event timestamp that is generated by the respective insulin pen upon occurrence of the insulin medicament injection event. In some embodiments, the data collection device 200 also receives glucose measurements from one or more glucose sensors (e.g., continuous glucose sensors) 102 used by the subject to measure glucose levels. In some embodiments, the data collection device 200 receives such data directly from the insulin pens 104 and/or glucose sensor(s) 102 and used by the subject. For instance, in some embodiments, the data collection device 200 receives this data wirelessly through radio-frequency signals. In some embodiments, such signals are in accordance with an 802.11 (WiFi), Bluetooth, or ZigBee standard. In some embodiments, the data collection device 200 receives such data directly, analyzes the data, and passes the analyzed data to the regimen dosage device 250. In some embodiments, an insulin pen 104 and/or a glucose sensor 102 includes an RFID tag and communicates to the data collection device 200 and/or the regimen dosage device 250 using RFID communication. In some embodiments, the data collection device 200 also obtains or receives physiological measurements of the subject (e.g., from wearable physiological measurement devices, from measurement devices within the data collection device 200 such as a magnetometer or a thermostat, etc.).

In some embodiments, the data collection device 200 and/or the regimen dosage device 250 is not proximate to the subject and/or does not have wireless capabilities or such wireless capabilities are not used for the purpose of acquiring insulin medicament injection data, autonomous glucose data, and/or physiological measurement data. In such embodiments, a communication network 106 may be used to communicate insulin medicament injection data from the one or more insulin pens 104 to the data collection device 200 and/or the regimen dosage device 250, and/or autonomous glucose measurements from the glucose sensor 102 to the data collection device 200 and/or the regimen dosage device 250, and/or physiological measurement data from one or more physiological measurement devices (not shown) to the data collection device 200 and/or the regimen dosage device 250.

Examples of networks 106 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

In some embodiments, the data collection device 200 and/or the regimen dosage device 250 is part of an insulin pen. That is, in some embodiments, the data collection device 200 and/or the regimen dosage device 250 and an insulin pen 104 are a single device.

In some embodiments, there is a single glucose sensor 102 attached to the subject and the data collection device 200 and/or the regimen dosage device 250 is part of the glucose sensor 102. That is, in some embodiments, the data collection device 200 and/or the regimen dosage device 250 and the glucose sensor 102 are a single device.

Of course, other topologies of the system 48 are possible. For instance, rather than relying on a communications network 106, the one or more insulin pens 104 and the optional one or more glucose sensors 102 may wirelessly transmit information directly to the data collection device 200 and/or regimen dosage device 250. Further, the data collection device 200 and/or the regimen dosage device 250 may constitute a portable electronic device, a server computer, or in fact constitute several computers that are linked together in a network or be a virtual machine in a cloud computing context. As such, the exemplary topology shown in FIG. 1 merely serves to describe the features of an embodiment of the present disclosure in a manner that will be readily understood to one of skill in the art.

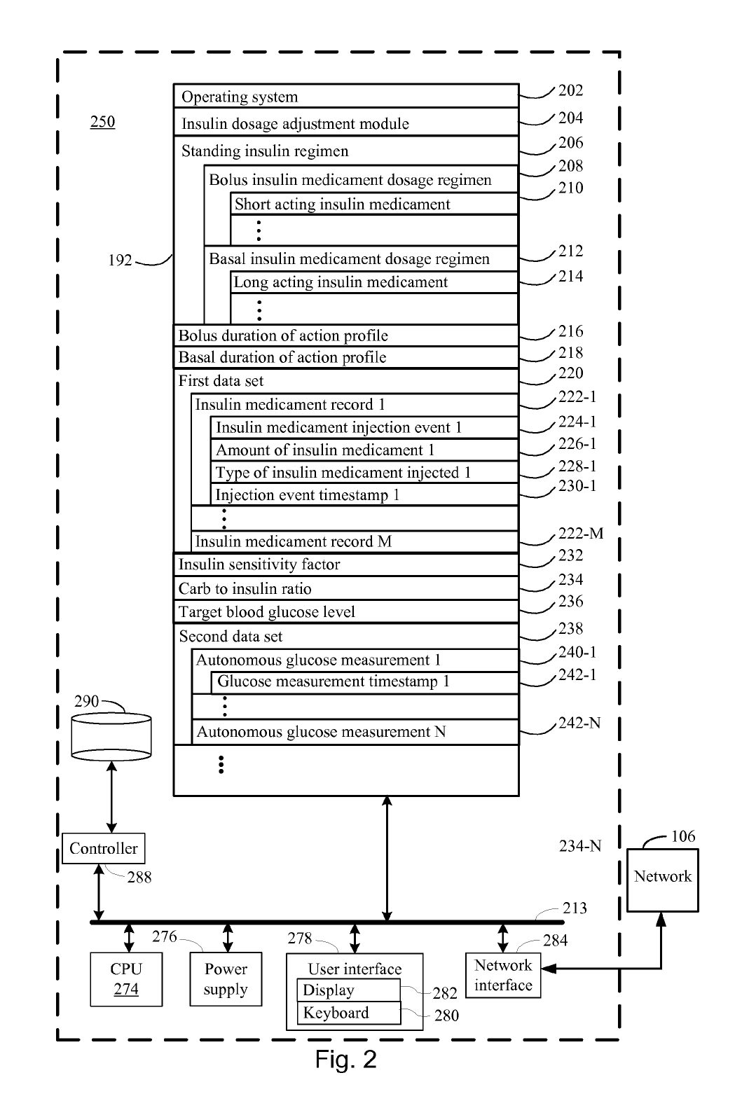

Referring to FIG. 2, in typical embodiments, the regimen dosage device 250 comprises one or more computers. For purposes of illustration in FIG. 2, the regimen dosage device 250 is represented as a single computer that includes all of the functionality for adjusting a short acting insulin medicament dosage (210) for a prospective meal event for a subject. However, the disclosure is not so limited. In some embodiments, the functionality for adjusting a short acting insulin medicament dosage (210) for a prospective meal event for a subject is spread across any number of networked computers and/or resides on each of several networked computers and/or is hosted on one or more virtual machines at a remote location accessible across the communications network 106. One of skill in the art will appreciate that any of a wide array of different computer topologies are used for the application and all such topologies are within the scope of the present disclosure.

Turning to FIG. 2 with the foregoing in mind, an exemplary regimen dosage device 250 for adjusting a short acting insulin medicament dosage for a prospective meal event for a subject comprises one or more processing units (CPU's) 274, a network or other communications interface 284, a memory 192 (e.g., random access memory), one or more magnetic disk storage and/or persistent devices 290 optionally accessed by one or more controllers 288, one or more communication busses 213 for interconnecting the aforementioned components, a user interface 278, the user interface 278 including a display 282 and input 280 (e.g., keyboard, keypad, touch screen), and a power supply 276 for powering the aforementioned components. In some embodiments, data in memory 192 is seamlessly shared with non-volatile memory 290 using known computing techniques such as caching. In some embodiments, memory 192 and/or memory 290 includes mass storage that is remotely located with respect to the central processing unit(s) 274. In other words, some data stored in memory 192 and/or memory 290 may in fact be hosted on computers that are external to the regimen dosage device 250 but that can be electronically accessed by the regimen dosage device 250 over an Internet, intranet, or other form of network or electronic cable (illustrated as element 106 in FIG. 2) using network interface 284.

In some embodiments, the memory 192 of the regimen dosage device 250 for adjusting a short acting insulin medicament dosage for a prospective meal event for a subject stores:
an operating system 202 that includes procedures for handling various basic system services;
an dosage adjustment module 204;
a standing insulin regimen 206 for the subject, the standing insulin regimen comprising (i) a bolus insulin medicament dosage regimen 208 comprising a short acting insulin medicament 210 and (ii) a basal insulin medicament dosage regimen 212 comprising a long acting insulin medicament 214;
a bolus duration of action profile 216 that indicates a duration of action of the short acting insulin medicament 210;
a basal duration of action profile 218 that indicates a duration of action of the long acting insulin medicament 214;
a first data set 220 comprising a plurality of insulin medicament records over a time course, each respective insulin medicament record 222 in the plurality of medicament records comprising: (i) a respective insulin medicament injection event 224 including an amount of insulin medicament 226 injected into the subject using a respective insulin pen 104 in the one or more insulin pens, (ii) a respective type of insulin medicament 228 injected into the subject from one of (a) the short acting insulin medicament and (b) the long acting insulin medicament, and (iii) a corresponding electronic injection event timestamp 230 that is automatically generated by a respective insulin pen upon occurrence of the respective insulin medicament injection event 224;
an insulin sensitivity factor 232 of the subject;
a carb to insulin ratio 234 for a prospective meal event;
a target blood glucose level 236 for the subject;
a second data set 238 that comprises a plurality of autonomous glucose measurements of the subject and, for each respective autonomous glucose measurement 240 in the plurality of autonomous glucose measurements, a glucose measurement timestamp 242 representing when the respective measurement was made.

In some embodiments, the insulin dosage adjustment module 204 is accessible within any browser (phone, tablet, laptop/desktop). In some embodiments the insulin dosage adjustment module 204 runs on native device frameworks, and is available for download onto the regimen dosage device 250 running an operating system 202 such as Android or iOS.

In some implementations, one or more of the above identified data elements or modules of the regimen dosage device 250 for adjusting a short acting insulin medicament dosage 210 for a prospective meal event for a subject are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 192 and/or 290 optionally stores a subset of the modules and data structures identified above. Furthermore, in some embodiments, the memory 192 and/or 290 stores additional modules and data structures not described above.

In some embodiments, a regimen dosage device 250 for adjusting a short acting insulin medicament dosage 210 for a prospective meal event for a subject is a smart phone (e.g., an iPHONE), laptop, tablet computer, desktop computer, or other form of electronic device (e.g., a gaming console). In some embodiments, the regimen dosage device 250 is not mobile. In some embodiments, the regimen dosage device 250 is mobile.

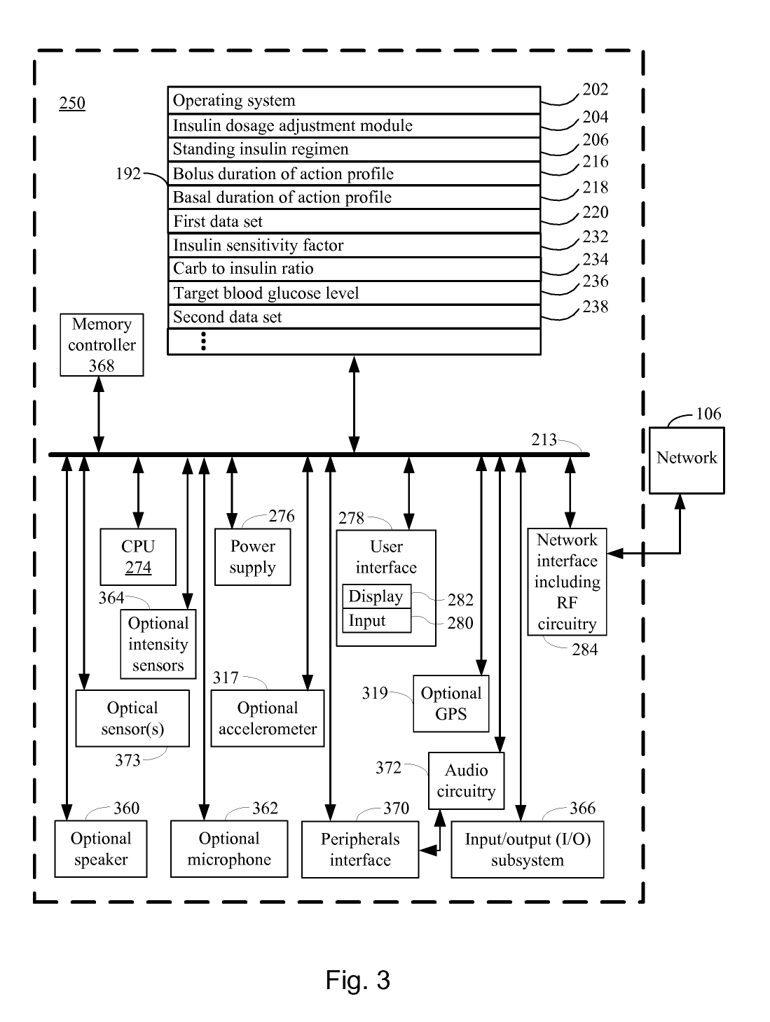

FIG. 3 provides a further description of a specific embodiment of a regimen dosage device 250 that can be used with the instant disclosure. The regimen dosage device 250 illustrated in FIG. 3 has one or more processing units (CPU's) 274, peripherals interface 370, memory controller 368, a network or other communications interface 284, a memory 192 (e.g., random access memory), a user interface 278, the user interface 278 including a display 282 and input 280 (e.g., keyboard, keypad, touch screen), an optional accelerometer 317, an optional GPS 319, optional audio circuitry 372, an optional speaker 360, an optional microphone 362, one or more optional intensity sensors 364 for detecting intensity of contacts on the regimen dosage device 250 (e.g., a touch-sensitive surface such as a touch-sensitive display system 282 of the regimen dosage device 250), an optional input/output (I/O) subsystem 366, one or more optional optical sensors 373, one or more communication busses 213 for interconnecting the aforementioned components, and a power supply 276 for powering the aforementioned components.

In some embodiments, the input 280 is a touch-sensitive display, such as a touch-sensitive surface. In some embodiments, the user interface 278 includes one or more soft keyboard embodiments. The soft keyboard embodiments may include standard (QWERTY) and/or non-standard configurations of symbols on the displayed icons.

The regimen dosage device 250 illustrated in FIG. 3 optionally includes, in addition to accelerometer(s) 317, a magnetometer (not shown) and a GPS 319 (or GLONASS or other global navigation system) receiver for obtaining information concerning the location and orientation (e.g., portrait or landscape) of the regimen dosage device 250 and/or for determining an amount of physical exertion by the subject.

It should be appreciated that the regimen dosage device 250 illustrated in FIG. 3 is only one example of a multifunction device that may be used for adjusting a short acting insulin medicament dosage (210) for a prospective meal event for a subject, and that the regimen dosage device 250 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Memory 192 of the regimen dosage device 250 illustrated in FIG. 3 optionally includes high-speed random access memory and optionally also includes non-volatile memory, such as one or more magnetic disk storage devices, flash memory devices, or other non-volatile solid-state memory devices. Access to memory 192 by other components of the regimen dosage device 250, such as CPU(s) 274 is, optionally, controlled by the memory controller 368.

The peripherals interface 370 can be used to couple input and output peripherals of the device to CPU(s) 274 and memory 192. The one or more processors 274 run or execute various software programs and/or sets of instructions stored in memory 192, such as the insulin dosage adjustment module 204, to perform various functions for the regimen dosage device 250 and to process data.

In some embodiments, the peripherals interface 370, CPU(s) 274, and memory controller 368 are, optionally, implemented on a single chip. In some other embodiments, they are implemented on separate chips.

RF (radio frequency) circuitry of network interface 284 receives and sends RF signals, also called electromagnetic signals. In some embodiments, the standing insulin regimen 206, the first data set 220, and/or the second data set 238 is received using this RF circuitry from one or more devices such as a glucose sensor 102 associated with a subject, an insulin pen 104 associated with the subject and/or the data collection device 200. In some embodiments, the RF circuitry 108 converts electrical signals to/from electromagnetic signals and communicates with communications networks and other communications devices, glucose sensors 102, and insulin pens 104 and/or the data collection device 200 via the electromagnetic signals. The RF circuitry 284 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 284 optionally communicates with the communication network 106. In some embodiments, the circuitry 284 does not include RF circuitry and, in fact, is connected to the network 106 through one or more hard wires (e.g., an optical cable, a coaxial cable, or the like).

In some embodiments, the audio circuitry 372, the optional speaker 360, and the optional microphone 362 provide an audio interface between the subject and the regimen dosage device 250. The audio circuitry 372 receives audio data from the peripherals interface 370, converts the audio data to electrical signals, and transmits the electrical signals to the speaker 360. The speaker 360 converts the electrical signals to human-audible sound waves. The audio circuitry 372 also receives electrical signals converted by the microphone 362 from sound waves. The audio circuitry 372 converts the electrical signal to audio data and transmits the audio data to peripherals interface 370 for processing. Audio data is, optionally, retrieved from and/or transmitted to the memory 192 and/or the RF circuitry 284 by the peripherals interface 370.

In some embodiments, the power supply 276 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

In some embodiments, the regimen dosage device 250 optionally also includes one or more optical sensors 373. The optical sensor(s) 373 optionally include charge-coupled device (CCD) or complementary metal-oxide semiconductor (CMOS) phototransistors. The optical sensor(s) 373 receive light from the environment, projected through one or more lens, and converts the light to data representing an image. The optical sensor(s) 373 optionally capture still images and/or video. In some embodiments, an optical sensor is located on the back of the regimen dosage device 250, opposite the display 282 on the front of the regimen dosage device 250, so that the input 280 is enabled for use as a viewfinder for still and/or video image acquisition. In some embodiments, another optical sensor 373 is located on the front of the regimen dosage device 250 so that the subject's image is obtained (e.g., to verify the health or condition of the subject, to determine the physical activity level of the subject, to help diagnose a subject's condition remotely, or to acquire visual physiological measurements of the subject, etc.).

As illustrated in FIG. 3, a regimen dosage device 250 preferably comprises an operating system 202 that includes procedures for handling various basic system services. The operating system 202 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

In some embodiments the regimen dosage device 250 is a smart phone. In other embodiments, the regimen dosage device 250 is not a smart phone but rather is a tablet computer, desktop computer, emergency vehicle computer, or other form or wired or wireless networked device. In some embodiments, the regimen dosage device 250 has any or all of the circuitry, hardware components, and software components found in the regimen dosage device 250 depicted in FIG. 2 or 3. In the interest of brevity and clarity, only a few of the possible components of the regimen dosage device 250 are shown in order to better emphasize the additional software modules that are installed on the regimen dosage device 250.

While the system 48 disclosed in FIG. 1 can work standalone, in some embodiments it can also be linked with electronic medical records to exchange information in any way.

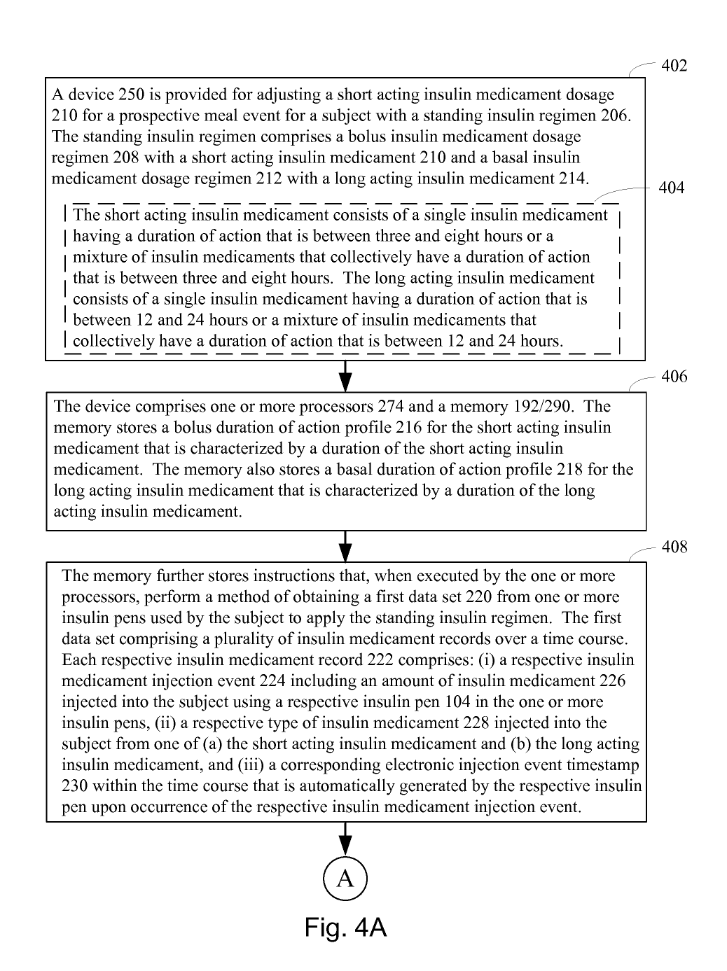

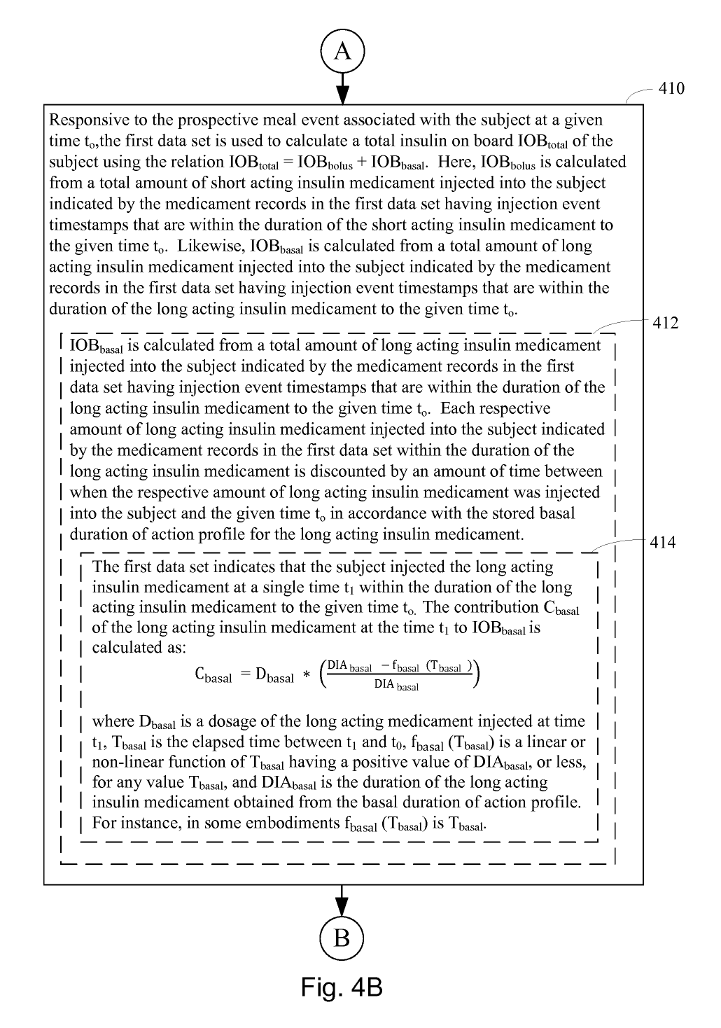

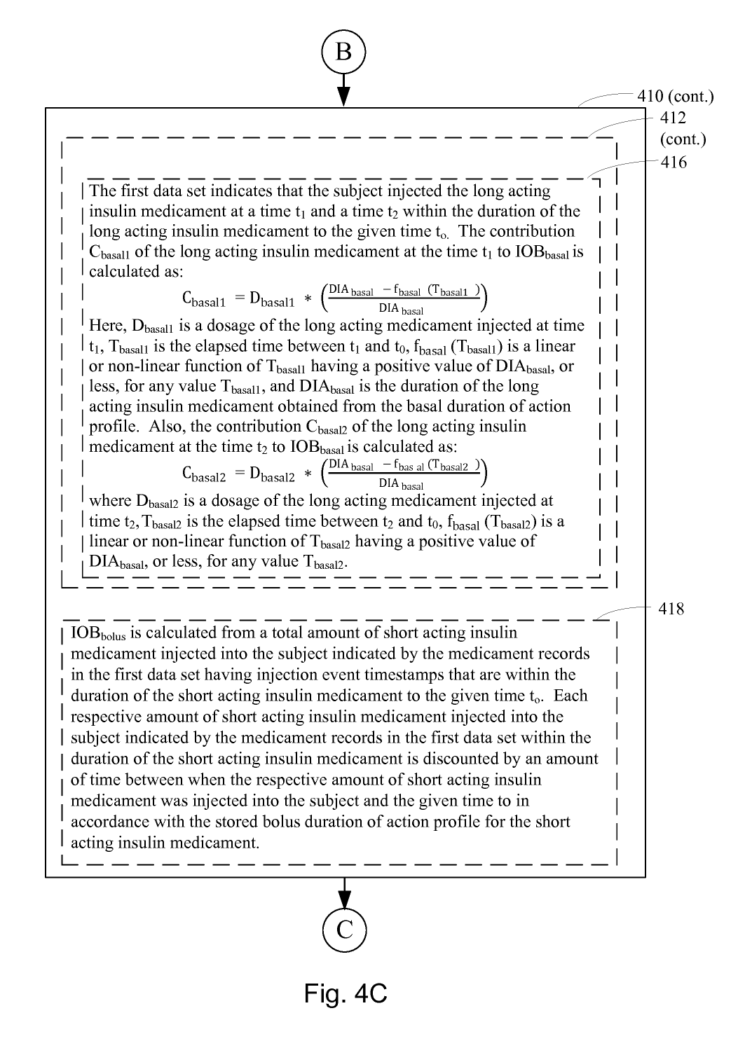

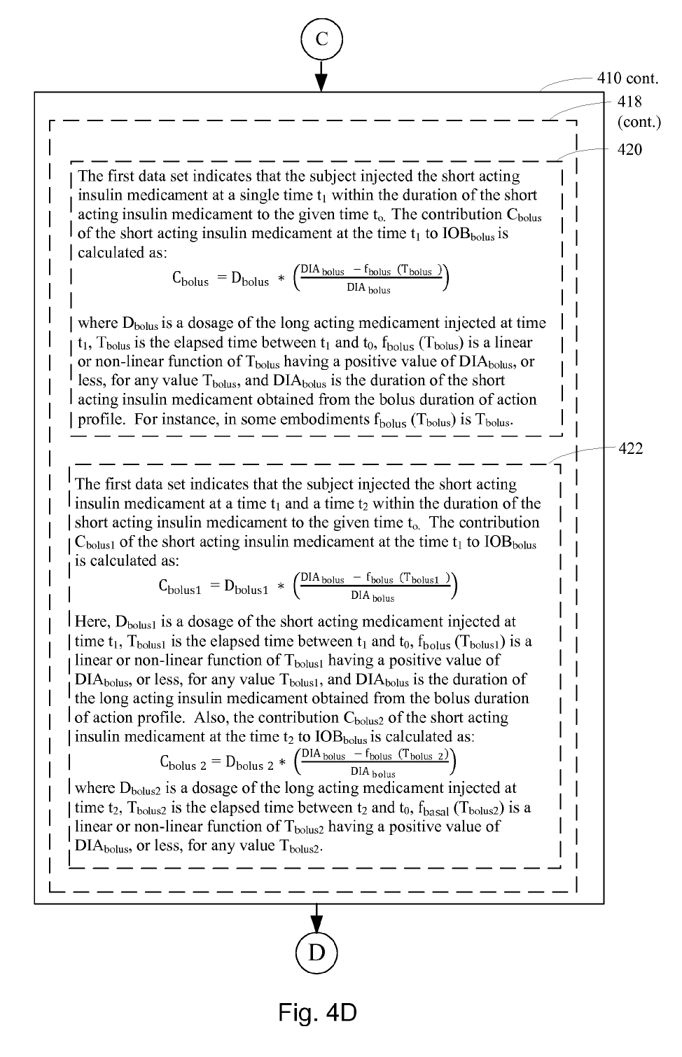

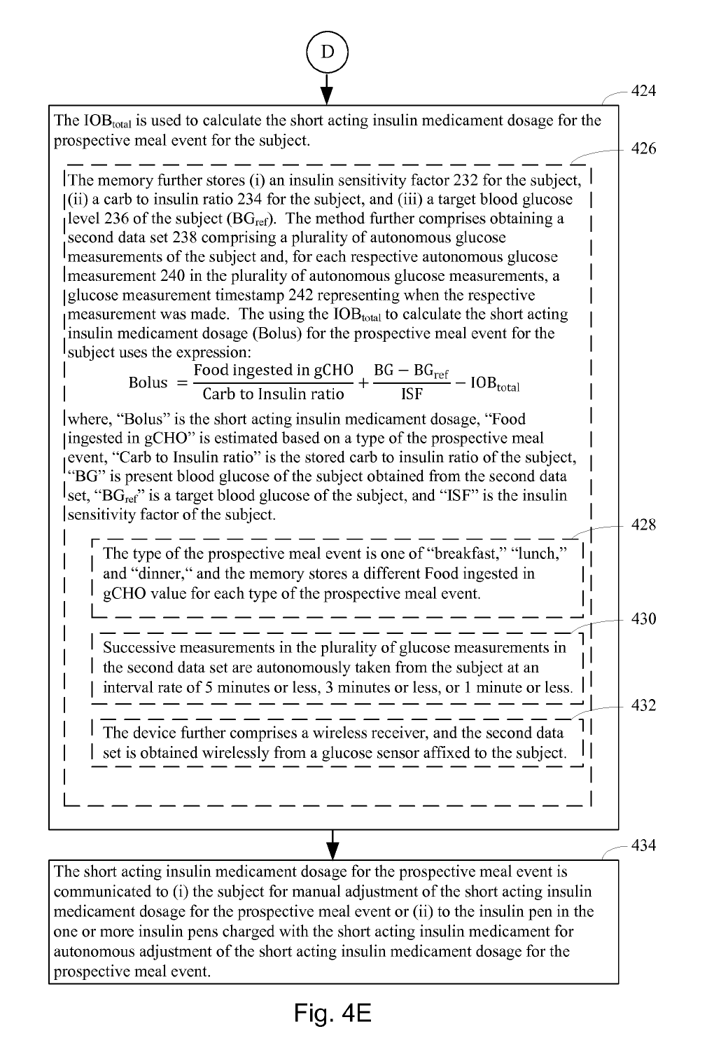

Now that details of a system 48 for adjusting a short acting insulin medicament dosage 210 for a prospective meal event for a subject have been disclosed, details regarding a flow chart of processes and features of the system, in accordance with an embodiment of the present disclosure, are disclosed with reference to FIGS. 4A through 4D. In some embodiments, such processes and features of the system are carried out by the insulin dosage adjustment module 204 illustrated in FIGS. 2 and 3.

Blocks 402-404.

With reference to block 402 of FIG. 4A, the goal of insulin therapy in subjects with either type 1 diabetes mellitus or type 2 diabetes mellitus is to match as closely as possible normal physiologic insulin secretion to control fasting and postprandial plasma glucose. As illustrated in FIG. 2, a device is 250 is provided for adjusting a short acting insulin medicament dosage 210 for a prospective meal event for a subject with a standing insulin regimen 206. The standing insulin regimen comprises a bolus insulin medicament dosage regimen 208 with a short acting insulin medicament 210 and a basal insulin medicament dosage regimen 212 with a long acting insulin medicament 214.

Referring to block 404 of FIG. 4A, in some embodiments, the short acting insulin medicament 210 consists of a single insulin medicament having a duration of action that is between three and eight hours or a mixture of insulin medicaments that collectively have a duration of action that is between three and eight hours. Examples of such short acting insulin medicaments include, but are not limited, to Lispro (HUMALOG, May 18, 2001, insulin lispro [rDNA origin] injection, Indianapolis, Ind.: Eli Lilly and Company), Aspart (NOVOLOG, July 2011, insulin aspart [rDNA origin] injection, Princeton, N.J., NOVO NORDISK Inc., July, 2011), Glulisine (Helms Kelley, 2009, “Insulin glulisine: an evaluation of its pharmacodynamic properties and clinical application,” Ann Pharmacother 43:658-668), and Regular (Gerich, 2002, “Novel insulins: expanding options in diabetes management,” Am J Med. 113:308-316).

In some embodiments, the long acting insulin medicament 214 consists of a single insulin medicament having a duration of action that is between 12 and 24 hours or a mixture of insulin medicaments that collectively have a duration of action that is between 12 and 24 hours. Examples of such long acting insulin medicaments include, but are not limited to, Insulin Degludec (developed by NOVO NORDISK under the brand name Tresiba), NPH (Schmid, 2007, “New options in insulin therapy,” J Pediatria (Rio J). 83(Suppl 5): S146-S155), Glargine (LANTUS, Mar. 2, 2007), Insulin Glargine [rDNA origin] injection (Dunn et al. 2003, “An Updated Review of its Use in the Management of Diabetes Mellitus” Drugs 63: p. 1743), and Determir (Plank et al., 2005, “A double-blind, randomized, dose-response study investigating the pharmacodynamic and pharmacokinetic properties of the long-acting insulin analog detemir,” Diabetes Care 28:1107-1112).

Block 406.

Referring to block 406 of FIG. 4A, the regimen dosage device 250 comprises one or more processors 274 and a memory 192/290. The memory stores a bolus duration of action profile 216 for the short acting insulin medicament 210 that is characterized by a duration of the short acting insulin medicament. The memory also stores a basal duration of action profile 218 for the long acting insulin medicament 214 that is characterized by a duration of the long acting insulin medicament.

Block 408.

Referring to block 406 of FIG. 4A, the memory further stores instructions that, when executed by the one or more processors 274, perform a method of obtaining a first data set 220 from one or more insulin pens 104 used by the subject to apply the standing insulin regimen 206. The first data set 220 comprises a plurality of insulin medicament records over a time course. Each respective insulin medicament record 222 comprises: (i) a respective insulin medicament injection event 224 including an amount of insulin medicament 226 injected into the subject using a respective insulin pen 104 in the one or more insulin pens, (ii) a respective type of insulin medicament 228 injected into the subject from one of (a) the short acting insulin medicament 210 and (b) the long acting insulin medicament 214, and (iii) a corresponding electronic injection event timestamp 230 within the time course that is automatically generated by the respective insulin pen 104 upon occurrence of the respective insulin medicament injection event.

Blocks 410-422.

At block 410 of FIG. 4B, a situation arises in which the insulin dosage adjustment module 204 is alerted to a prospective meal event. For instance, in some embodiments, the subject may toggle an affordance (e.g., interactive radio button) in a graphical user interface provided by the insulin dosage adjustment module 204. In some such embodiments, the user may specify parameters of the prospective meal event such as an estimated amount of carbohydrates in the prospective meal event and optionally the glycaemic index of these carbohydrates, the fat content of the prospective meal event, and/or the size (e.g., in calories) of the prospective meal event. In other embodiments, prospective meal events are calendared and not input from the user is required. In an example of such an embodiment, when the subject eats breakfast, lunch and dinner during the day is calendared and the insulin dosage adjustment module 204 is triggered into action by each of these calendared meals. The alert to the insulin dosage adjustment module 204 for the prospective meal includes an estimated or actual time of occurrence of the prospective meal to. Regardless of the mechanism by which the insulin dosage adjustment module 204 is alerted to the prospective meal event, responsive to the prospective meal event associated with the subject, occurring or estimated to occur at the given time tₒ, the acquired first data set 220 is used to calculate a total insulin on board IOBₜₒₜₐₗ of the subject using the relation IOBₜₒₜₐₗ=IOB_{bolus}+IOB_{basal}.

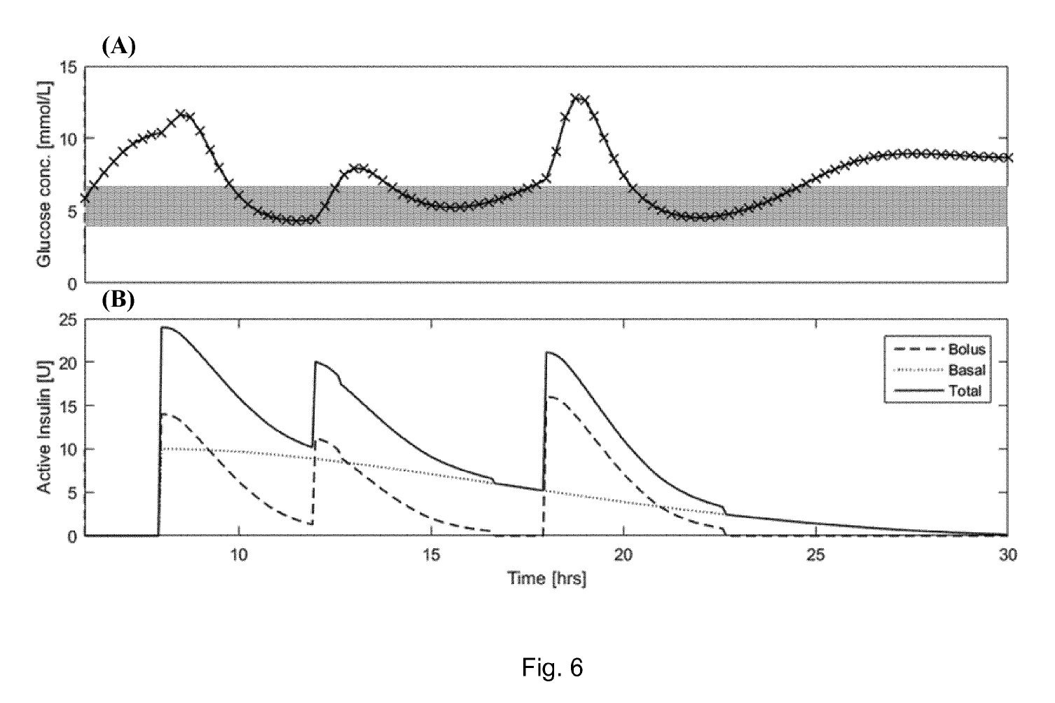

FIG. 6 illustrates. FIG. 6, top panel (A), illustrates glucose concentration over a 30 hour period during which three meals are ingested with corresponding short acting insulin medicament injections (boluses) and one long acting insulin medicament (basal) injection, whereas FIG. 6, bottom panel (B), shows an estimation of active insulin units at every point in time, for short acting insulin medicament insulin, long acting insulin medicament insulin, and their total, in accordance with an embodiment of the present disclosure. If insulin pen data for basal and bolus insulin injections are available, the present disclosure can estimate how many units are active of each type of insulin on board (IOB) (bolus and basal) at any given point in time, given the drugs PK/PD profile. FIG. 6 illustrates how the total active IOB is estimated during a period of 24 hours, where three bolus and one basal injections are taken.

As such, IOB_{bolus} is calculated from a total amount of short acting insulin medicament 210 injected into the subject using an insulin pen 104 as indicated by the insulin medicament records 222 in the first data set 220 having injection event timestamps 230 that are within the duration of the short acting insulin medicament 210 to the given time tₒ. For instance, consider the case in which the bolus duration of action profile 216 indicates that the duration of the short acting insulin medicament 210 is 30 minutes and that the time tₒ of the prospective meal event on a given day is noon. In such embodiments, any insulin medicament records 222 in the first data set 220 that include a short acting insulin medicament 210 injection event (type of insulin medicament injected 228=the short acting insulin medicament 210) with an injection event timestamp 230 between 11:30 AM and 12:30 PM will contribute to IOB_{bolus}. In some embodiments two or more short acting insulin medicament 210 injection events contribute to IOB_{bolus}.

Likewise, IOB_{basal} is calculated from a total amount of long acting insulin medicament 214 injected into the subject indicated by the insulin medicament records in the first data set 220 having injection event timestamps 230 that are within the duration of the long acting insulin medicament 214 to the given time tₒ. For instance, consider the case in which the basal duration of action profile 218 indicates that the duration of the long acting insulin medicament 214 is 6 hours and that the time tₒ of the prospective meal event on a given day is noon. In such embodiments, any insulin medicament records 222 in the first data set 220 that include a long acting insulin medicament 214 injection event (type of insulin medicament injected 228=the long acting insulin medicament 214) with an injection event timestamp 230 between 6:00 AM and 6:00 PM will contribute to IOB_{basal}. In some embodiments two or more long acting insulin medicament 214 injection events contribute to IOB_{basal}.

Referring to block 412 of FIG. 4B, in some embodiments, the amount of time between a respective long acting insulin medicament 214 injection event and the time tₒ of the prospective meal event serves to discount the amount the respective long acting insulin medicament 214 injection event contributes to IOB_{basal}. In such embodiments IOB_{basal} is calculated from a total amount of long acting insulin medicament 214 injected into the subject as indicated by the insulin medicament records 222 in the first data set 220 having injection event timestamps 230 that are within the duration of the long acting insulin medicament to the given time tₒ of the prospective meal event. However, each respective amount of long acting insulin medicament 214 injected into the subject indicated by the insulin medicament records 222 in the first data set 220 within the duration of the long acting insulin medicament in such embodiments is discounted by an amount of time between when the respective amount of long acting insulin medicament 214 was injected into the subject (as indicated by the corresponding injection event timestamp 230) and the given time tₒ of the prospective meal event in accordance with the stored basal duration of action profile 218 for the long acting insulin medicament.

Block 414 of FIG. 4B illustrates one way in which the amount of time between the basal injection event and the time tₒ of the prospective meal event is discounted in some such embodiments. In block 414, the first data set 220 indicates that the subject injected the long acting insulin medicament 214 at a single time t₁ within the duration of the long acting insulin medicament (specified by the basal duration profile 218) to the given time tₒ of the prospective meal event. The contribution C_{basal} of the long acting insulin medicament at the time t₁ to IOB_{basal} is then calculated as:

$${C}_{basal} = {D}_{basal} * ( \frac{{DIA}_{basal} - {f}_{basal} ( {T}_{basal} )}{{DIA}_{basal}} )$$

where D_{basal} is a dosage of the long acting insulin medicament 214 injected at time t₁, T_{basal} is the elapsed time between t₁ and t₀, f_{basal}(T_{basal}) is a linear or non-linear function (e.g., a polynomial function, a power series, a logarithmic function, an exponential function, a series expansion of an exponential or logarithmic function, a Taylor series, an ordinary differential equation, etc.) of T_{basal} having a positive value that is DIA_{basal}, or less (but always greater than zero), for any value T_{basal}, and DIA_{basal} is the duration of the long acting insulin medicament obtained from the basal duration of action profile 218. For instance, in some embodiments f_{basal}(T_{basal}) is T_{basal}. In other words, in some embodiments f_{basal}(T_{basal}) is simply the value of T_{basal}. In some embodiments, the linear or non-linear function of T_{basal} accounts for drop off in the drug action of the long acting insulin medicament 214 over time by accounting for one or more characteristics of the long acting insulin medicament 214 such as: the absolute amount of long acting insulin medicament 214 given, the pharmaceutical preparation of the long acting insulin medicament 214, the half-life of the long acting insulin medicament 214, and/or the slope of the concentration-response curve of the long acting insulin medicament 214 as determined in a clinical trial or other published work. In some embodiments, f_{basal}(T_{basal}) is computed using a published dose response curve for the long acting insulin medicament 214. That is, a published dose response curve for the long acting insulin medicament 214, stored in basal duration of action profile 218 (or otherwise electronically accessible to the insulin dosage adjustment module 204) is used to model f_{basal}(T_{basal}) such that the equation:

$${C}_{basal} = {D}_{basal} * ( \frac{{DIA}_{basal} - {f}_{basal} ( {T}_{basal} )}{{DIA}_{basal}} )$$

accurately reflects C_{basal} as a function of the parameters D_{basal} and the T_{basal}.

It will be appreciated that the equation given in block 414 is merely exemplary and that any equation for C_{basal} for a given basal injection event occurring within the duration of the long acting insulin medicament to tₒ of the prospective meal event that discounts the contribution of the injection event to C_{basal} on a temporal basis by accounting for the absolute amount of long acting insulin medicament 214 injected in the basal injection event, the pharmaceutical preparation of the long acting insulin medicament 214, the half-life of the long acting insulin medicament 214, the slope of the concentration-response curve of the long acting insulin medicament 214 and/or other pharmacokinetic properties of the long acting insulin medicament 214 is within the scope of the present disclosure.

Block 416 of FIG. 4C extends the discussion of such discounting of contributions to C_{basal} as a function of time to the case where there are two basal injection events occurring within the duration of the long acting insulin medicament to tₒ of the prospective meal event. In this exemplary embodiment, the first data set 220 indicates that the subject injected the long acting insulin medicament at a time t₁ and at a time t₂ within the duration of the long acting insulin medicament to the given time tₒ of the prospective meal event. The contribution C_{basal1} of the long acting insulin medicament at the time t₁ to IOB_{basal} is calculated as:

$${C}_{basal 1} = {D}_{basal 1} * ( \frac{{DIA}_{basal} - {f}_{basal} ( {T}_{basal 1} )}{{DIA}_{basal}} )$$

Here, D_{basal1} is a dosage of the long acting medicament 214 injected at time t₁, T_{basal1} is the elapsed time between t₁ and tₒ, f_{basal}(T_{basal1}) is a linear or non-linear function (e.g., a polynomial function, a power series, a logarithmic function, an exponential function, a series expansion of an exponential or logarithmic function, a Taylor series, an ordinary differential equation, etc.) of T_{basal1} having a positive value of DIA_{basal}, or less (but always greater than zero), for any value T_{basal1}, and DIA_{basal} is the duration of the long acting insulin medicament obtained from the basal duration of action profile. In some embodiments f_{basal}(T_{basal1}) is T_{basal}. In other words, in some embodiments f_{basal}(T_{basal1}) is simply the value of T_{basal}. Also, the contribution C_{basal2} of the long acting insulin medicament at the time t₂ to IOB_{basal} is calculated as:

$${C}_{basal 2} = {D}_{basal 2} * ( \frac{{DIA}_{basal} - {f}_{basal} ( {T}_{basal 2} )}{{DIA}_{basal}} )$$

where D_{basal2} is a dosage of the long acting medicament injected at time t₂, T_{basal2} is the elapsed time between t₂ and tₒ, f_{basal}(T_{basal2}) is a linear or non-linear function (e.g., a polynomial function, a power series, a logarithmic function, an exponential function, a series expansion of an exponential or logarithmic function, a Taylor series, an ordinary differential equation, etc.) of T_{basal2} having a positive value of DIA_{basal}, or less, for any value T_{basal2}.

It will be appreciated that the equation given in block 416 is merely exemplary and that any equation for C_{basal} for a given combination of basal injection events occurring within the duration of the long acting insulin medicament to tₒ of the prospective meal event that discounts the contribution of the injection events to C_{basal} on a temporal basis by accounting for the absolute amount of long acting insulin medicament 214 injected in each of the basal injection events, the pharmaceutical preparation of the long acting insulin medicament 214, the half-life of the long acting insulin medicament 214, the slope of the concentration-response curve of the long acting insulin medicament 214 and/or other pharmacokinetic properties of the long acting insulin medicament 214 is within the scope of the present disclosure.

Referring to block 418 of FIG. 4C, as was the corresponding case for IOB_{bolus} in block 412, in some embodiments, the amount of time between a respective short acting insulin medicament 210 injection event and the time tₒ of the prospective meal event serves to discount the amount the respective short acting insulin medicament 210 injection event contributes to IOB_{bolus}. Thus, in some embodiments, IOB_{bolus} is calculated from a total amount of short acting insulin medicament 210 injected into the subject indicated by the insulin medicament records in the first data set 220 having injection event timestamps 230 that are within the duration of the short acting insulin medicament to the given time tₒ of the prospective meal event. Each respective amount of short acting insulin medicament injected into the subject indicated by the insulin medicament records 222 in the first data set 220 within the duration of the short acting insulin medicament 210 is discounted by an amount of time between when the respective amount of short acting insulin medicament 210 was injected into the subject and the given time tₒ in accordance with the stored bolus duration of action profile 216 for the short acting insulin medicament.

Block 420 of FIG. 4D illustrates one way in which the amount of time between the bolus injection event and the time tₒ of the prospective meal event is discounted in some such embodiments. In block 420, the first data set 220 indicates that the subject injected the short acting insulin medicament 210 at a single time t₁ within the duration of the short acting insulin medicament (specified by the bolus duration profile 216) to the given time tₒ of the prospective meal event. The contribution C_{bolus} of the short acting insulin medicament at the time t₁ to IOB_{bolus} is then calculated as:

$${C}_{bolus} = {D}_{bolus} * ( \frac{{DIA}_{bolus} - {f}_{bolus} ( {T}_{bolus} )}{{DIA}_{bolus}} )$$

where D_{bolus1} is a dosage of the short acting insulin medicament 210 injected at time t₁, T_{bolus} is the elapsed time between t₁ and tₒ, f_{bolus}(T_{bolus}) is a linear or non-linear function (e.g., a polynomial function, a power series, a logarithmic function, an exponential function, a series expansion of an exponential or logarithmic function, a Taylor series, an ordinary differential equation, etc.) of T_{bolus} having a positive value that is DIA_{bolus}, or less (but always greater than zero), for any value T_{bolus}, and DIA_{bolus} is the duration of the short acting insulin medicament obtained from the bolus duration of action profile 216. For instance, in some embodiments f_{bolus}(T_{bolus}) is T_{bolus}. In other words, in some embodiments f_{bolus}(T_{bolus}) is simply the value of T_{bolus}. In some embodiments, the linear or non-linear function of T_{bolus} accounts for drop off in the drug action of the short acting insulin medicament 210 over time by accounting for one or more characteristics of the short acting insulin medicament 210 such as: the absolute amount of short acting insulin medicament 210 given in the respective injection, the pharmaceutical preparation of the short acting insulin medicament 210, the half-life of the short acting insulin medicament 210, and/or the slope of the concentration-response curve of the short acting insulin medicament 210 as determined in a clinical trial or other published work. In some embodiments, f_{bolus}(T_{bolus}) is computed using published dose response curve for the short acting insulin medicament 210. That is, a published dose response curve for the short acting insulin medicament 210, stored in bolus duration of action profile 216 (or otherwise electronically accessible to the insulin dosage adjustment module 204), is used to model f_{bolus}(T_{bolus}) such that the equation:

$${C}_{bolus} = {D}_{bolus} * ( \frac{{DIA}_{bolus} - {f}_{bolus} ( {T}_{bolus} )}{{DIA}_{bolus}} )$$

accurately reflects C_{bolus} as a function of the parameters D_{bolus} and the T_{bolus}.

It will be appreciated that the equation given in block 420 is merely exemplary and that any equation for C_{bolus} for a given basal injection event occurring within the duration of the short acting insulin medicament to tₒ of the prospective meal event that discounts the contribution of the injection event to C_{bolus} on a temporal basis by accounting for the absolute amount of short acting insulin medicament 210 injected in the bolus injection event, the pharmaceutical preparation of the short acting insulin medicament 210, the half-life of the short acting insulin medicament 210, the slope of the concentration-response curve of the short acting insulin medicament 210 and/or other pharmacokinetic properties of the short acting insulin medicament 210 is within the scope of the present disclosure.

Block 422 of FIG. 4D extends the discussion of such discounting of contributions to C_{bolus} as a function of time to the case where there are two bolus injection events occurring within the duration of the short acting insulin medicament to tₒ of the prospective meal event. In this exemplary embodiment, the first data set 220 indicates that the subject injected the short acting insulin medicament at a time t₁ and at a time t₂ within the duration of the short acting insulin medicament to the given time tₒ of the prospective meal event. The contribution C_{bolus1} of the short acting insulin medicament at the time t₁ to IOB_{bolus} is calculated as:

$${C}_{bolus 1} = {D}_{bolus 1} * ( \frac{{DIA}_{bolus} - {f}_{bolus} ( {T}_{bolus 1} )}{{DIA}_{bolus}} )$$

Here, D_{bolus1} is a dosage of the short acting medicament 210 injected at time t₁, T_{bolus1} is the elapsed time between t₁ and t₀, f_{bolus}(T_{bolus1}) is a linear or non-linear function (e.g., a polynomial function, a power series, a logarithmic function, an exponential function, a series expansion of an exponential or logarithmic function, a Taylor series, an ordinary differential equation, etc.) of T_{bolus1} having a positive value of DIA_{bolus}, or less (but always greater than zero), for any value T_{bolus1}, and DIA_{bolus} is the duration of the short acting insulin medicament obtained from the bolus duration of action profile. In some embodiments f_{bolus}(T_{bolus1}) is T_{bolus}. In other words, in some embodiments f_{bolus1}(T_{bolus1}) is simply the value of T_{bolus}. Also, the contribution C_{bolus2} of the short acting insulin medicament at the time t₂ to IOB_{bolus} is calculated as:

$${C}_{bolus 2} = {D}_{bolus 2} * ( \frac{{DIA}_{bolus} - {f}_{bolus} ( {T}_{bolus 2} )}{{DIA}_{bolus}} )$$

where D_{bolus2} is a dosage of the long acting medicament injected at time t₂, T_{bolus2} is the elapsed time between t₂ and tₒ, f_{basal}(T_{bolus2}) is a linear or non-linear function (e.g., a polynomial function, a power series, a logarithmic function, an exponential function, a series expansion of an exponential or logarithmic function, a Taylor series, an ordinary differential equation, etc.) of T_{bolus2} having a positive value of DIA_{bolus}, or less, for any value T_{bolus2}.

It will be appreciated that the equation given in block 422 is merely exemplary and that any equation for C_{bolus} for a given combination of bolus injection events occurring within the duration of the short acting insulin medicament to tₒ of the prospective meal event that discounts the contribution of the injection events to C_{bolus} on a temporal basis by accounting for the absolute amount of short acting insulin medicament 210 injected in each of the bolus injection events, the pharmaceutical preparation of the short acting insulin medicament 210, the half-life of the short acting insulin medicament 210, the slope of the concentration-response curve of the short acting insulin medicament 210, and/or other pharmacokinetic properties of the long acting insulin medicament 210 is within the scope of the present disclosure.

Blocks 424-432.

In block 424 of FIG. 4E, the method continues by using IOBₜₒₜₐₗ, computed above, to calculate the short acting insulin medicament dosage for the prospective meal event for the subject.

For instance, referring to block 426 of FIG. 4E, in some embodiments, the memory 192/290 further stores (i) an insulin sensitivity factor 232 for the subject, (ii) a carb to insulin ratio 234 for the subject, and (iii) a target blood glucose level 236 of the subject (BG_{ref}). In such embodiments, the method further comprises obtaining a second data set 238 comprising a plurality of autonomous glucose measurements of the subject and, for each respective autonomous glucose measurement 240 in the plurality of autonomous glucose measurements, a glucose measurement timestamp 242 representing when the respective measurement was made. In typical embodiments, these autonomous glucose measurements are from one or more glucose sensors 102. FIG. 2 illustrates. Each such autonomous glucose measurement 240 is timestamped with a glucose measurement timestamp 242 to represent when the respective measurement was made. Thus, in typical embodiments, the autonomous glucose measurements are measured without human intervention. That is, the subject does not manually make the autonomous glucose measurements. In alternative embodiments of the present disclosure, the subject or a health care practitioner manually takes glucose measurements and such manual glucose measurements are used as a substitute or a complement to the autonomous glucose measurements 240 in the second data set 238.

In embodiments where autonomous glucose measurements are used, devices such as the FREESTYLE LIBRE CGM by ABBOTT (“LIBRE”) may serve as the glucose sensor 102 in order to make the plurality of autonomous glucose measurements of a subject. The LIBRE allows calibration-free glucose measurements with an on-skin coin-sized sensor, which can send up to eight hours of data to a reader device (e.g., the data collection device 200 and/or the regimen dosage device 250) via near field communications, when brought close together. The LIBRE can be worn for fourteen days in all daily life activities. In some embodiments, the autonomous glucose measurements are autonomously taken from the subject at an interval rate of 5 minutes or less, 3 minutes or less, or 1 minute or less. In some embodiments, the autonomous glucose measurements are taken from the subject at an interval rate of 5 minutes or less, 3 minutes or less, or 1 minute or less, over a time period of a day or more, two days or more, a week or more, or two weeks or more. In some embodiments, the autonomous glucose measurements are autonomously taken (e.g., without human effort, without human intervention, etc.).

In some embodiments, IOBₜₒₜₐₗ derived as discussed above, is used to calculate the short acting insulin medicament dosage (Bolus) for the prospective meal event for the subject using the expression:

$$Bolus = \frac{Food ingested in gCHO}{Carb to Insulin ratio} + \frac{BG - {BG}_{ref}}{ISF} - {IOB}_{total}$$

where, “Bolus” is the short acting insulin medicament dosage to be calculated, “Food ingested in gCHO” is estimated based on a type of the prospective meal event, “Carb to Insulin ratio” is the stored carb to insulin ratio of the subject, “BG” is present blood glucose of the subject obtained from the second data set, “BG_{ref}” is a target blood glucose of the subject, and “ISF” is the insulin sensitivity factor of the subject.

It will be appreciated that the equation given in block 426 is merely exemplary and that any equation for calculating “Bolus” that takes into account IOBₜₒₜₐₗ is within the scope of the present disclosure. Such equations for calculating “Bolus” may consider any number of factors in addition to IOBₜₒₜₐₗ. For instance, in some embodiments, such equations consider “Food ingested in gCHO” (estimated based on a type of the prospective meal event), “Carb to Insulin ratio” (the stored carb to insulin ratio of the subject), “BG” (present blood glucose of the subject obtained from the second data set), “BG_{ref}” (target blood glucose of the subject), and “ISF” (insulin sensitivity factor of the subject). In some embodiments, such equations consider any two or more, three or more or four or more of the factors: “Food ingested in gCHO” (estimated based on a type of the prospective meal event), “Carb to Insulin ratio” (the stored carb to insulin ratio of the subject), “BG” (present blood glucose of the subject obtained from the second data set), “BG_{ref}” (target blood glucose of the subject), and “ISF” (insulin sensitivity factor of the subject), an estimated amount of carbohydrates in the prospective meal event, glycaemic index of these carbohydrates, the fat content of the prospective meal event, and the size (e.g., in calories) of the prospective meal event.

Referring to block 430, in some embodiments, the type of the prospective meal event is one of “breakfast,” “lunch,” and “dinner,” and the memory 192/290 stores a different “Food ingested in gCHO value” for each type of the prospective meal event. Referring to block 432, in some embodiments, the device further comprises a wireless receiver, and the second data set is obtained wirelessly from a glucose sensor affixed to the subject.

Block 434.

In block 434 of FIG. 4E, the method continues with the communication of the short acting insulin medicament dosage for the prospective meal event to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event. Advantageously, communication of the short acting insulin medicament dosage for the prospective meal event allows the subject to optimize the amount of short acting insulin medicament dosage relative to meal events in between health care practitioner visits.

### Example

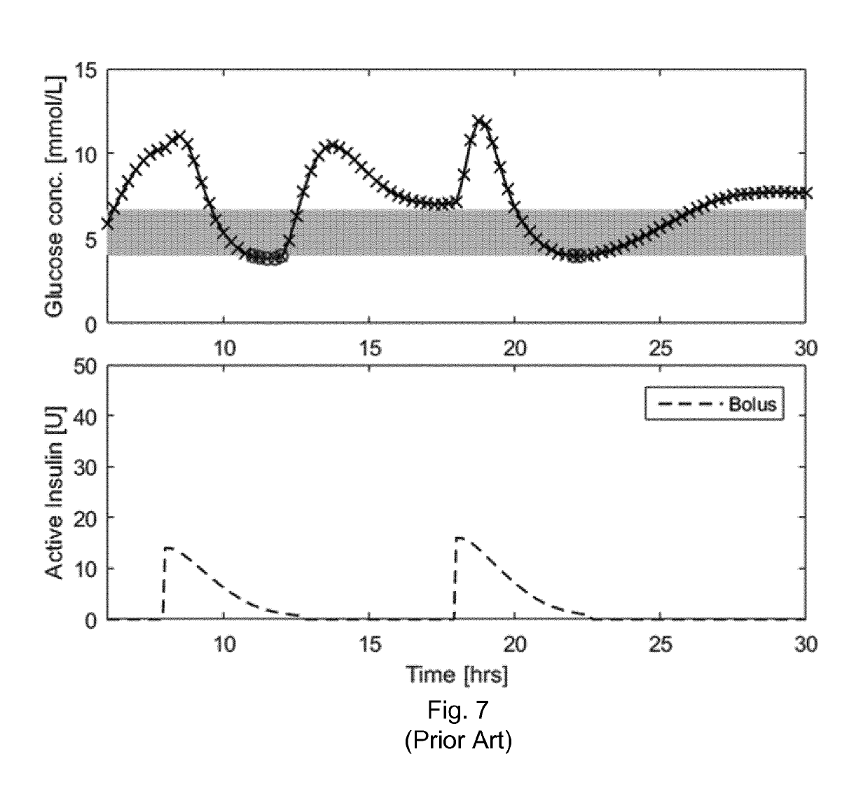

FIG. 7 illustrates a situation where the patient takes basal insulin in the morning, but only bolus IOB is accounted for when determining a bolus size. Since the algorithm does not know that a large dose of basal was taken at 7:00, a full bolus is calculated to compensate for the meal and high blood sugar at 8:00 (this is the traditional way of calculating bolus insulin):

$$Bolus = Meal Bolus + Hyperglycaemia Bolus - IOB = \frac{Food ingested in gCHO}{Carb to Insulin ratio} + \frac{BG - {BG}_{ref}}{Insulin Sensitivity Factor} - {IOB}_{bolus}$$

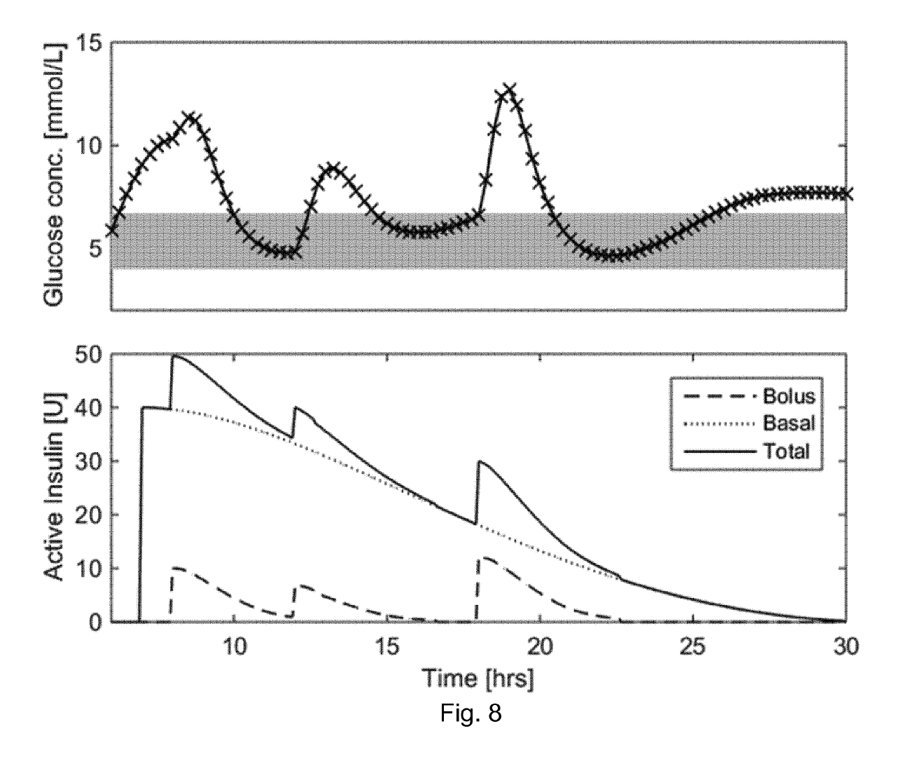

However, the basal injection compensates for part of the high blood sugar, and therefore the bolus taken at 8:00 is too large, and causes hypoglycaemia (indicated by circles).
FIG. 8 illustrates a similar situation, but now the systems and method of the present disclosure are compute the total insulin on board estimate, and accounts for that when determining the size of a bolus injection:

$$Bolus = Meal Bolus + Hyperglycaemia Bolus - IOB = \frac{Food ingested in gCHO}{Carb to Insulin ratio} + \frac{BG - {BG}_{ref}}{Insulin Sensitivity Factor} - {IOB}_{bolus} - a * {IOB}_{basal}$$

where a indicates the percentage of basal units to subtract from the bolus. Hence, at 8:00, the systems and methods of the present disclosure know that a large basal injection was taken at 7:00 and subtracts a percentage of the units given as basal.

### LIST OF EMBODIMENTS

1. A device 250 for adjusting a short acting insulin medicament dosage 210 for a prospective meal event for a subject with a standing insulin regimen 206, wherein

the standing insulin regimen comprises a bolus insulin medicament dosage regimen 208 with a short acting insulin medicament 210 and a basal insulin medicament dosage regimen 212 with a long acting insulin medicament 214;

the device comprises one or more processors 274 and a memory 192/290, the memory storing:
a bolus duration of action profile 216 for the short acting insulin medicament that is characterized by a duration of the short acting insulin medicament, and
a basal duration of action profile 218 for the long acting insulin medicament that is characterized by a duration of the long acting insulin medicament,

the memory further storing instructions that, when executed by the one or more processors, perform a method of:

obtaining a first data set 220 from one or more insulin pens used by the subject to apply the standing insulin regimen, the first data set comprising a plurality of insulin medicament records over a time course, each respective insulin medicament record 222 in the plurality of medicament records comprising:
(i) a respective insulin medicament injection event 224 including an amount of insulin medicament 226 injected into the subject using a respective insulin pen 104 in the one or more insulin pens,
(ii) a respective type of insulin medicament 228 injected into the subject from one of (a) the short acting insulin medicament and (b) the long acting insulin medicament, and
(iii) a corresponding electronic injection event timestamp 230 within the time course that is automatically generated by the respective insulin pen upon occurrence of the respective insulin medicament injection event; and

responsive to receiving an indication of the prospective meal event associated with the subject at a given time tₒ:
using the first data set to calculate a total insulin on board IOBₜₒₜₐₗ of the subject, wherein the IOBₜₒₜₐₗ is calculated using the relation:

IOBₜₒₜₐₗ=IOB_{bolus}+IOB_{basal}
wherein,
   IOB_{bolus} is calculated from a total amount of short acting insulin medicament injected into the subject indicated by the medicament records in the first data set having injection event timestamps that are within the duration of the short acting insulin medicament to the given time tₒ, and
   IOB_{basal} is calculated from a total amount of long acting insulin medicament injected into the subject indicated by the medicament records in the first data set having injection event timestamps that are within the duration of the long acting insulin medicament to the given time tₒ;
using the IOBₜₒₜₐₗ to calculate the short acting insulin medicament dosage for the prospective meal event for the subject; and
communicating the short acting insulin medicament dosage for the prospective meal event to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event.

2. The device of embodiment 1, wherein
the memory further stores (i) an insulin sensitivity factor 232 for the subject, (ii) a carb to insulin ratio 234 for the subject, and (iii) a target blood glucose level 236 of the subject (BG_{ref}), and
the method further comprises:
   obtaining a second data set 238, the second data set comprising a plurality of autonomous glucose measurements of the subject and, for each respective autonomous glucose measurement 240 in the plurality of autonomous glucose measurements, a glucose measurement timestamp 242 representing when the respective measurement was made, and wherein
   the using the IOBₜₒₜₐₗ to calculate the short acting insulin medicament dosage (Bolus) for the prospective meal event for the subject using the expression:

$$Bolus = \frac{Food ingested in gCHO}{Carb to Insulin ratio} + \frac{BG - {BG}_{ref}}{ISF} - {IOB}_{total}$$

wherein,
   Bolus is the short acting insulin medicament dosage,
   Food ingested in gCHO is estimated based on a type of the prospective meal event,
   Carb to Insulin ratio is the stored carb to insulin ratio of the subject,
   BG is present blood glucose of the subject obtained from the second data set,
   BG_{ref} is a target blood glucose of the subject, and
   ISF is the insulin sensitivity factor of the subject.

3. The device of embodiment 2, wherein the type of the prospective meal event is one of “breakfast,” “lunch,” and “dinner,” and wherein the memory stores a different Food ingested in gCHO value for each type of the prospective meal event.

4. The device of any one of embodiments 1-3, wherein
IOB_{basal} is calculated from a total amount of long acting insulin medicament injected into the subject indicated by the medicament records in the first data set having injection event timestamps that are within the duration of the long acting insulin medicament to the given time tₒ, and
each respective amount of long acting insulin medicament injected into the subject indicated by the medicament records in the first data set within the duration of the long acting insulin medicament is discounted by an amount of time between when the respective amount of long acting insulin medicament was injected into the subject and the given time tₒ in accordance with the stored basal duration of action profile for the long acting insulin medicament.

5. The device of embodiment 4, wherein
the first data set indicates that the subject injected the long acting insulin medicament at a single time t₁ within the duration of the long acting insulin medicament to the given time tₒ, and the contribution C_{basal} of the long acting insulin medicament at the time t₁ to IOB_{basal} is calculated as:

$${C}_{basal} = {D}_{basal} * ( \frac{{DIA}_{basal} - {f}_{basal} ( {T}_{basal} )}{{DIA}_{basal}} )$$

wherein,
   D_{basal} is a dosage of the long acting medicament injected at time t₁,
   T_{basal} is the elapsed time between t₁ and t₀,
   f_{basal}(T_{basal}) is a linear or non-linear function of T_{basal} having a positive value of DIA_{basal}, or less, for any value T_{basal}, and
   DIA_{basal} is the duration of the long acting insulin medicament obtained from the basal duration of action profile.

6. The device of embodiment 5, wherein f_{basal}(T_{basal}) is T_{basal}.

7. The device of embodiment 4, wherein
the first data set indicates that the subject injected the long acting insulin medicament at a time t₁ and a time t₂ within the duration of the long acting insulin medicament to the given time tₒ, and
the contribution C_{basal1} of the long acting insulin medicament at the time t₁ to IOB_{basal} is calculated as:

$${C}_{basal 1} = {D}_{basal 1} * ( \frac{{DIA}_{basal} - {f}_{basal} ( {T}_{basal 1} )}{{DIA}_{basal}} )$$

wherein,
   D_{basal1} is a dosage of the long acting medicament injected at time t₁,
   T_{basal1} is the elapsed time between t₁ and t₀,
   f_{basal}(T_{basal1}) is a linear or non-linear function of T_{basal1} having a positive value of DIA_{basal}, or less, for any value T_{basal1}, and
   DIA_{basal} is the duration of the long acting insulin medicament obtained from the basal duration of action profile, and
the contribution C_{basal2} of the long acting insulin medicament at the time t₂ to IOB_{basal} is calculated as:

$${C}_{basal 2} = {D}_{basal} * ( \frac{{DIA}_{basal} - {f}_{basal} ( {T}_{basal 2} )}{{DIA}_{basal}} )$$

wherein,
   D_{basal2} is a dosage of the long acting medicament injected at time t₂,
   T_{basal} is the elapsed time between t₂ and tₒ, and
   f_{basal}(T_{basal2}) is a linear or non-linear function of T_{basal2} having a positive value of DIA_{basal}, or less, for any value T_{basal2}.

8. The device of any one of embodiments 1-6, wherein
IOB_{bolus} is calculated from a total amount of short acting insulin medicament injected into the subject indicated by the medicament records in the first data set having injection event timestamps that are within the duration of the short acting insulin medicament to the given time tₒ,
each respective amount of short acting insulin medicament injected into the subject indicated by the medicament records in the first data set within the duration of the short acting insulin medicament is discounted by an amount of time between when the respective amount of short acting insulin medicament was injected into the subject and the given time tₒ in accordance with the stored bolus duration of action profile for the short acting insulin medicament.

9. The device of embodiment 8, wherein
the first data set indicates that the subject injected the short acting insulin medicament at a single time t₃ within the duration of the short acting insulin medicament to the given time tₒ, and
the contribution C_{bolus} of the short acting insulin medicament at the time t₃ to IOB_{bolus} is calculated as:

$${C}_{bolus} = {D}_{bolus} * ( \frac{{DIA}_{bolus} - {f}_{bolus} ( {T}_{bolus} )}{{DIA}_{bolus}} )$$

wherein,
   D_{bolus} is a dosage of the short acting medicament injected at time t₃,
   T_{bolus} is the elapsed time between t₃ and t₀,
   f_{bolus}(T_{bolus}) is a linear or non-linear function of T_{bolus} having a positive value of DIA_{bolus}, or less, for any value T_{bolus}, and
   DIA_{bolus} is the duration of the short acting insulin medicament obtained from the bolus duration of action profile.

10. The device of embodiment 9, wherein f_{bolus}(T_{bolus}) is T_{bolus}.

11. The device of embodiment 8, wherein
the first data set indicates that the subject injected the short acting insulin medicament at a first time t₃ and a second time t₄ within the duration of the short acting insulin medicament to the given time tₒ, and
the contribution C_{bolus1} of the short acting insulin medicament at the time t₃ to IOB_{bolus} is calculated as:

$${C}_{bolus 1} = {D}_{bolus 1} * ( \frac{{DIA}_{bolus} - {f}_{bolus} ( {T}_{bolus 1} )}{{DIA}_{bolus}} )$$

wherein,
   D_{bolus1} is a dosage of the short acting medicament injected at time t₃,
   T_{bolus1} is the elapsed time between t₃ and t₀,
   f_{bolus}(T_{bolus1}) is a linear or non-linear function of T_{bolus1} having a positive value of DIA_{bolus}, or less, for any value T_{bolus1}, and
   DIA_{bolus} is the duration of the short acting insulin medicament obtained from the bolus duration of action profile, and
the contribution C_{bolus2} of the short acting insulin medicament at the time t₄ to IOB_{bolus} is calculated as:

$${C}_{bolus 2} = {D}_{bolus 2} * ( \frac{{DIA}_{bolus} - {f}_{bolus} ( {T}_{bolus 2} )}{{DIA}_{bolus}} )$$

wherein,
   D_{bolus2} is a dosage of the short acting medicament injected at time t₄,
   T_{bolus2} is the elapsed time between t₄ and t₀, and
   f_{bolus}(T_{bolus2}) is a linear or non-linear function of T_{bolus2} having a positive value of DIA_{bolus}, or less, for any value T_{bolus2}.

12. The device of embodiment 2, wherein successive measurements in the plurality of autonomous glucose measurements in the second data set are autonomously taken from the subject at an interval rate of 5 minutes or less, 3 minutes or less, or 1 minute or less.

13. The device of embodiment 2, wherein the device further comprises a wireless receiver, and wherein the second data set is obtained wirelessly from a glucose sensor affixed to the subject.

14. The device of any one of embodiments 1-13, wherein
the short acting insulin medicament consists of a single insulin medicament having a duration of action that is between three and eight hours or a mixture of insulin medicaments that collectively have a duration of action that is between three and eight hours, and
the long acting insulin medicament consists of a single insulin medicament having a duration of action that is between 12 and 24 hours or a mixture of insulin medicaments that collectively have a duration of action that is between 12 and 24 hours.

15. A method for adjusting a short acting insulin medicament dosage for a prospective meal event for a subject with a standing insulin regimen, the memory comprising:
at a computer comprising one or more processors and a memory:
the memory storing:
   a standing insulin regimen, wherein the standing insulin regimen comprises a bolus insulin medicament dosage regimen with a short acting insulin medicament and a basal insulin medicament dosage regimen with a long acting insulin medicament,
   a bolus duration of action profile for the short acting insulin medicament that is characterized by a duration of the short acting insulin medicament, and
   a basal duration of action profile for the long acting insulin medicament that is characterized by a duration of the long acting insulin medicament,
the memory further storing instructions that, when executed by the one or more processors, perform a method of:
obtaining a first data set from one or more insulin pens used by the subject to apply the standing insulin regimen, the first data set comprising a plurality of insulin medicament records over a time course, each respective insulin medicament record in the plurality of medicament records comprising:
   (i) a respective insulin medicament injection event including an amount of insulin medicament injected into the subject using a respective insulin pen in the one or more insulin pens,
   (ii) a respective type of insulin medicament injected into the subject from one of (a) the short acting insulin medicament and (b) the long acting insulin medicament, and
   (iii) a corresponding electronic injection event timestamp within the time course that is automatically generated by the respective insulin pen upon occurrence of the respective insulin medicament injection event; and
responsive to receiving an indication of the prospective meal event associated with the subject at a given time tₒ:
   using the first data set to calculate a total insulin on board IOBₜₒₜₐₗ of the subject, wherein the IOBₜₒₜₐₗ is calculated using the relation:

IOBₜₒₜₐₗ=IOB_{bolus}+IOB_{basal}
wherein,
   IOB_{bolus} is calculated from a total amount of short acting insulin medicament injected into the subject indicated by the medicament records in the plurality of medicament records having injection event timestamps that are within the duration of the short acting insulin medicament to the given time tₒ, and
   IOB_{basal} is calculated from a total amount of long acting insulin medicament injected into the subject indicated by the medicament records in the first data set having injection event timestamps that are within the duration of the long acting insulin medicament to the given time tₒ;
using the IOBₜₒₜₐₗ to calculate the short acting insulin medicament dosage for the prospective meal event for the subject; and
communicating the short acting insulin medicament dosage for the prospective meal event to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event.

16. A computer program comprising instructions that, when executed by one or more processors, perform the method of embodiment 15.

17. A computer-readable data carrier having stored thereon the computer program according to embodiment 16.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a nontransitory computer readable storage medium. For instance, the computer program product could contain the program modules shown in any combination of FIGS. 1, 2, 3, 5 and/or described in FIG. 4. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A device for adjusting a short acting insulin medicament dosage for a prospective meal event for a subject with a standing insulin regimen, wherein
the standing insulin regimen comprises a bolus insulin medicament dosage regimen with a short acting insulin medicament and a basal insulin medicament dosage regimen with a long acting insulin medicament;
the device comprises one or more processors and a memory, the memory storing:
a bolus duration of action profile for the short acting insulin medicament that is characterized by a duration of the short acting insulin medicament, and
a basal duration of action profile for the long acting insulin medicament that is characterized by a duration of the long acting insulin medicament,
the memory further storing instructions that, when executed by the one or more processors, perform a method of:
obtaining a first data set from one or more insulin pens used by the subject to apply the standing insulin regimen, the first data set comprising a plurality of insulin medicament records over a time course, each respective insulin medicament record in the plurality of medicament records comprising:
(i) a respective insulin medicament injection event including an amount of insulin medicament injected into the subject using a respective insulin pen in the one or more insulin pens,
(ii) a respective type of insulin medicament injected into the subject from one of (a) the short acting insulin medicament and (b) the long acting insulin medicament, and
(iii) a corresponding electronic injection event timestamp within the time course that is automatically generated by the respective insulin pen upon occurrence of the respective insulin medicament injection event; and
responsive to receiving an indication of the prospective meal event associated with the subject at a given time tₒ:
using the first data set to calculate a total insulin on board IOBₜₒₜₐₗ of the subject, wherein the IOBₜₒₜₐₗ is calculated using the relation:
IOBₜₒₜₐₗ=IOB_{bolus}+IOB_{basal}
wherein,
IOB_{bolus} is calculated from a total amount of short acting insulin medicament injected into the subject indicated by the medicament records in the first data set having injection event timestamps that are within the duration of the short acting insulin medicament to the given time tₒ, and
IOB_{basal} is calculated from a total amount of long acting insulin medicament injected into the subject indicated by the medicament records in the first data set having injection event timestamps that are within the duration of the long acting insulin medicament to the given time tₒ;
using the IOBₜₒₜₐₗ to calculate the short acting insulin medicament dosage for the prospective meal event for the subject; and
communicating the short acting insulin medicament dosage for the prospective meal event to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event.

2. The device of claim 1, wherein
the memory further stores:
(i) an insulin sensitivity factor for the subject,
(ii) a carb to insulin ratio for the subject, and
(iii) a target blood glucose level of the subject (BG_{ref}), and
the method further comprises:
obtaining a second data set, the second data set comprising a plurality of autonomous glucose measurements of the subject and, for each respective autonomous glucose measurement in the plurality of autonomous glucose measurements, a glucose measurement timestamp representing when the respective measurement was made, and wherein
the using the IOBₜₒₜₐₗ to calculate the short acting insulin medicament dosage (Bolus) for the prospective meal event for the subject using the expression:
$$Bolus = \frac{Food ingested in gCHO}{Carb to Insulin ratio} + \frac{BG - {BG}_{ref}}{ISF} - {IOB}_{total}$$
wherein,
Bolus is the short acting insulin medicament dosage,
Food ingested in gCHO is estimated based on a type of the prospective meal event,
Carb to Insulin ratio is the stored carb to insulin ratio of the subject,
BG is present blood glucose of the subject obtained from the second data set,
BG_{ref} is a target blood glucose of the subject, and
ISF is the insulin sensitivity factor of the subject.

3. The device of claim 2, wherein the type of the prospective meal event is one of “breakfast,” “lunch,” and “dinner,” and wherein the memory stores a different Food ingested in gCHO value for each type of the prospective meal event.

4. The device of claim 1, wherein
IOB_{basal} is calculated from a total amount of long acting insulin medicament injected into the subject indicated by the medicament records in the first data set having injection event timestamps that are within the duration of the long acting insulin medicament to the given time tₒ, and
each respective amount of long acting insulin medicament injected into the subject indicated by the medicament records in the first data set within the duration of the long acting insulin medicament is discounted by an amount of time between when the respective amount of long acting insulin medicament was injected into the subject and the given time tₒ in accordance with the stored basal duration of action profile for the long acting insulin medicament.

5. The device of claim 4, wherein
the first data set indicates that the subject injected the long acting insulin medicament at a single time t₁ within the duration of the long acting insulin medicament to the given time tₒ, and the contribution C_{basal} of the long acting insulin medicament at the time t₁ to IOB_{basal} is calculated as:
$${C}_{basal} = {D}_{basal} * ( \frac{{DIA}_{basal} - {f}_{basal} ( {T}_{basal} )}{{DIA}_{basal}} )$$
wherein,
D_{basal} is a dosage of the long acting medicament injected at time t₁,
T_{basal} is the elapsed time between t₁ and t₀,
f_{basal}(T_{basal}) is a linear or non-linear function of T_{basal} having a positive value of DIA_{basal}, or less, for any value T_{basal}, and
DIA_{basal} is the duration of the long acting insulin medicament obtained from the basal duration of action profile.

6. The device of claim 5, wherein f_{basal}(T_{basal}) is T_{basal}.

7. The device of claim 4, wherein
the first data set indicates that the subject injected the long acting insulin medicament at a time t₁ and a time t₂ within the duration of the long acting insulin medicament to the given time tₒ, and
the contribution C_{basal1} of the long acting insulin medicament at the time t₁ to IOB_{basal} is calculated as:
$${C}_{basal 1} = {D}_{basal 1} * ( \frac{{DIA}_{basal} - {f}_{basal} ( {T}_{basal 1} )}{{DIA}_{basal}} )$$
wherein,
D_{basal1} is a dosage of the long acting medicament injected at time t₁,
T_{basal1} is the elapsed time between t₁ and t₀,
f_{basal}(T_{basal1}) is a linear or non-linear function of T_{basal1} having a positive value of DIA_{basal}, or less, for any value T_{basal1}, and
DIA_{basal} is the duration of the long acting insulin medicament obtained from the basal duration of action profile, and
the contribution C_{basal2} of the long acting insulin medicament at the time t₂ to IOB_{basal} is calculated as:
$${C}_{basal 2} = {D}_{basal 2} * ( \frac{{DIA}_{basal} - {f}_{basal} ( {T}_{basal 2} )}{{DIA}_{basal}} )$$
wherein,
D_{basal2} is a dosage of the long acting medicament injected at time t₂,
T_{basal2} is the elapsed time between t₂ and t₀, and
f_{basal} is a linear or non-linear function of T_{basal2} having a positive value of DIA_{basal}, or less, for any value T_{basal2}.

8. The device of claim 1, wherein
IOB_{bolus} is calculated from a total amount of short acting insulin medicament injected into the subject indicated by the medicament records in the first data set having injection event timestamps that are within the duration of the short acting insulin medicament to the given time tₒ,
each respective amount of short acting insulin medicament injected into the subject indicated by the medicament records in the first data set within the duration of the short acting insulin medicament is discounted by an amount of time between when the respective amount of short acting insulin medicament was injected into the subject and the given time tₒ in accordance with the stored bolus duration of action profile for the short acting insulin medicament.

9. The device of claim 8, wherein
the first data set indicates that the subject injected the short acting insulin medicament at a single time t₃ within the duration of the short acting insulin medicament to the given time tₒ, and
the contribution C_{bolus} of the short acting insulin medicament at the time t₃ to IOB_{bolus} is calculated as:
$${C}_{bolus} = {D}_{bolus} * ( \frac{{DIA}_{bolus} - {f}_{bolus} ( {T}_{bolus} )}{{DIA}_{bolus}} )$$
wherein,
D_{bolus} is a dosage of the short acting medicament injected at time t₃,
T_{bolus} is the elapsed time between t₃ and t₀,
f_{bolus}(T_{bolus}) is a linear or non-linear function of T_{bolus} having a positive value of DIA_{bolus} or less, for any value T_{bolus}, and
DIA_{bolus} is the duration of the short acting insulin medicament obtained from the bolus duration of action profile.

10. The device of claim 9, wherein f_{bolus}(T_{bolus}) is T_{bolus}.

11. The device of claim 8, wherein
the first data set indicates that the subject injected the short acting insulin medicament at a first time t₃ and a second time t₄ within the duration of the short acting insulin medicament to the given time tₒ, and
the contribution C_{bolus1} of the short acting insulin medicament at the time t₃ to IOB_{bolus} is calculated as:
$${C}_{bolus 1} = {D}_{bolus 1} * ( \frac{{DIA}_{bolus} - {f}_{bolus} ( {T}_{bolus 1} )}{{DIA}_{bolus}} )$$
wherein,
D_{bolus1} is a dosage of the short acting medicament injected at time t₃,
T_{bolus1} is the elapsed time between t₃ and t₀,
f_{bolus}(T_{bolus1}) is a linear or non-linear function of T_{bolus1} having a positive value of DIA_{bolus}, or less, for any value T_{bolus1}, and
DIA_{bolus} is the duration of the short acting insulin medicament obtained from the bolus duration of action profile, and
the contribution C_{bolus2} of the short acting insulin medicament at the time t₄ to IOB_{bolus} is calculated as:
$${C}_{bolus 2} = {D}_{bolus 2} * ( \frac{{DIA}_{bolus} - {f}_{bolus} ( {T}_{bolus 2} )}{{DIA}_{bolus}} )$$
wherein,
D_{bolus2} is a dosage of the short acting medicament injected at time t₄,
T_{bolus2} is the elapsed time between t₄ and t₀, and
f_{bolus}(T_{bolus2}) is a linear or non-linear function of T_{bolus2} having a positive value of DIA_{bolus}, or less, for any value T_{bolus2}.

12. The device of claim 2, wherein successive measurements in the plurality of autonomous glucose measurements in the second data set are autonomously taken from the subject at an interval rate of 5 minutes or less, 3 minutes or less, or 1 minute or less.

13. The device of claim 2, wherein the device further comprises a wireless receiver, and wherein the second data set is obtained wirelessly from a glucose sensor affixed to the subject.

14. The device of claim 1, wherein
the short acting insulin medicament consists of a single insulin medicament having a duration of action that is between three and eight hours or a mixture of insulin medicaments that collectively have a duration of action that is between three and eight hours, and
the long acting insulin medicament consists of a single insulin medicament having a duration of action that is between 12 and 24 hours or a mixture of insulin medicaments that collectively have a duration of action that is between 12 and 24 hours.

15. A method for adjusting a short acting insulin medicament dosage for a prospective meal event for a subject with a standing insulin regimen, the memory comprising:
at a computer comprising one or more processors and a memory:
the memory storing:
a standing insulin regimen, wherein the standing insulin regimen comprises a bolus insulin medicament dosage regimen with a short acting insulin medicament and a basal insulin medicament dosage regimen with a long acting insulin medicament,
a bolus duration of action profile for the short acting insulin medicament that is characterized by a duration of the short acting insulin medicament, and
a basal duration of action profile for the long acting insulin medicament that is characterized by a duration of the long acting insulin medicament,
the memory further storing instructions that, when executed by the one or more processors, perform a method of:
obtaining a first data set from one or more insulin pens used by the subject to apply the standing insulin regimen, the first data set comprising a plurality of insulin medicament records over a time course, each respective insulin medicament record in the plurality of medicament records comprising:
(i) a respective insulin medicament injection event including an amount of insulin medicament injected into the subject using a respective insulin pen in the one or more insulin pens,
(ii) a respective type of insulin medicament injected into the subject from one of (a) the short acting insulin medicament and (b) the long acting insulin medicament, and
(iii) a corresponding electronic injection event timestamp within the time course that is automatically generated by the respective insulin pen upon occurrence of the respective insulin medicament injection event; and
responsive to receiving an indication of the prospective meal event associated with the subject at a given time tₒ:
using the first data set to calculate a total insulin on board IOBₜₒₜₐₗ of the subject, wherein the IOBₜₒₜₐₗ is calculated using the relation:
IOBₜₒₜₐₗ=IOB_{bolus}+IOB_{basal}
wherein,
IOB_{bolus} is calculated from a total amount of short acting insulin medicament injected into the subject indicated by the medicament records in the plurality of medicament records having injection event timestamps that are within the duration of the short acting insulin medicament to the given time tₒ, and
IOB_{basal} is calculated from a total amount of long acting insulin medicament injected into the subject indicated by the medicament records in the first data set having injection event timestamps that are within the duration of the long acting insulin medicament to the given time tₒ;
using the IOBₜₒₜₐₗ to calculate the short acting insulin medicament dosage for the prospective meal event for the subject; and
communicating the short acting insulin medicament dosage for the prospective meal event to (i) the subject for manual adjustment of the short acting insulin medicament dosage for the prospective meal event or (ii) to the insulin pen in the one or more insulin pens charged with the short acting insulin medicament for autonomous adjustment of the short acting insulin medicament dosage for the prospective meal event.

16. A computer program comprising instructions that, when executed by a computer having one or more processors and a memory, perform the method of claim 15.

17. A computer-readable data carrier having stored thereon the computer program according to claim 16.
